SEQUENCE LISTING (4 sequences)

SEQ ID NO: 1 - protein, 491 aa, Homo sapiens [sequence omitted]

SEQ ID NO: 2 - protein, 491 aa, Mus musculus
MNAAAEAEFNILLATDSYKVTHYKQYPPNTSKVYSYFECREKKTENSKVRKVKYEETVFYGLQYILNKYLKGKVVTKEKIQEAKEVYREHFQDDVFNERGWNYILEKYDGHLPIEVKAVPEGSVIPRGNVLFTVENTDPECYWLTNWIETILVQSWYPITVATNSREQKKILAKYLLETSGNLDGLEYKLHDFGYRGVSSQETAGIGASAHLVNFKGTDTVAGIALIKKYYGTKDPVPGYSVPAAEHSTITAWGKDHEKDAFEHIVTQFSSVPVSVVSDSYDIYNACEKIWGEDLRHLIVSRSTEAPLIIRPDSGNPLDTVLKVLDILGKKFPVTENSKGYKLLPPYLRVIQGDGVDINTLQEIVEGMKQKKWSIENVSFGSGGALLQKLTRDLLNCSFKCSYVVTNGLGVNVFKDPVADPNKRSKKGRLSLHRTPAGNFVTLEEGKGDLEEYGHDLLHTVFKNGKVTKSYSFDEVRKNAQLNIEQDVAPH

SEQ ID NO: 3 - protein, 392 aa, Thermoplasma acidophilum
MNVFNTASDEDIKKGLASDVYFERTISAIGDKCNDLRVAMEATVSGPLDTWINFTGLDEVLKLLEGLDVDLYAIPEGTILFPRDANGLPVPFIRVEGRYCDFGMYETAILGFICQASGISTKASKVRLAAGDSPFFSFGIRRMHPAISPMIDRSAYIGGADGVSGILGAKLIDQDPVGTMPHALSIMLGDEEAWKLTLENTKNGQKSVLLIDTYMDEKFAAIKIAEMFDKVDYIRLDTPSSRRGNFEALIREVRWELALRGRSDIKIMVSGGLDENTVKKLREAGAEAFGVGTSISSAKPFDFAMDIVEVNGKPETKRGKMSGRKNVLRCTSCHRIEVVPANVQEKTCICGGSMQNLLVKYLSHGKRTSEYPRPKEIRSRSMKELEYFKDIS

SEQ ID NO: 4 - protein, 284 aa, Mycobacterium tuberculosis
GLSDWELAAARAAIARGLDEDLRYGPDVTTLATVPASATTTASLVTREAGVVAGLDVALLTLNEVLGTNGYRVLDRVEDGARVPPGEALMTLEAQTRGLLTAERTMLNLVGHLSGIATATAAWVDAVRGTKAKIRDTRKTLPGLRALQKYAVRTGGGVNHRLGLGDAALIKDNHVAAAGSVVDALRAVRNAAPDLPCEVEVDSLEQLDAVLPEKPELILLDNFAVWQTQTAVQRRDSRAPTVMLESSGGLSLQTAATYAETGVDYLAVGALTHSVRVLDIGLDM

(19) United States
(12) Patent Application Publication
(10) Pub. No.: US 2008/0020413 A1
(43) Pub. Date: Jan. 24, 2008

(54) CRYSTALLINE VISFATIN AND METHODS THEREFOR

(75) Inventors: Liang Tong, Scarsdale, NY (US); Xiao Tao, New York, NY (US); Javed A. Khan, Edison, NJ (US)

Correspondence Address: SONNENSCHEIN NATH & ROSENTHAL LLP, P.O. BOX 061080, WACKER DRIVE STATION, SEARS TOWER, CHICAGO, IL, 60606-1080, US

(73) Assignee: Columbia University, New York, NY (US)

(21) Appl. No.: 11/693,536

(22) Filed: Mar. 29, 2007

Related U.S. Application Data
(60) Provisional application No. 60/787,210, filed on Mar. 29, 2006.

(51) Int. Cl. C12N 9/98; C12N 9/96; C12Q 1/48; G06G 7/58

(52) U.S. Cl. 435/15; 435/187; 435/188; 703/11

(57) ABSTRACT

Crystals of nicotinamide phosphoribosyltransferase, methods of making the crystals, and methods of using the crystals are disclosed. The three-dimensional structures of NMPRTases are also disclosed. Also disclosed are methods for utilizing a crystal structure of an NMPRTase for identifying, designing, selecting, or testing molecules which affect NMPRTase activity, which can be used therapeutically in the treatment of diseases and disorders such as cancer and diabetes.

### CROSS-REFERENCE TO RELATED APPLICATIONS

This application claims priority from U.S. Provisional Application Ser. No. 60/787,210 filed on Mar. 29, 2006, which is incorporated herein by reference in its entirety.

### STATEMENT REGARDING FEDERALLY SPONSORED RESEARCH OR DEVELOPMENT

The disclosed teachings were developed in part with Government support under National Institutes of Health Grant DK67238. The Government has certain rights in the invention.

### INCORPORATION-BY-REFERENCE OF MATERIAL SUBMITTED ON A COMPACT DISC

Tables of atomic coordinates of protein crystals, which are part of the present disclosure, are included herein in a computer readable form. The subject matter of the tables (Table 1, Table 2, Table 3 and Table 4) are incorporated herein by reference in their entireties. File sizes are as follows: Table 1: 1,815 KB; Table 2: 653 KB; Table 3: 641 KB; Table 4: 1,114 KB.

LENGTHY TABLES FILED ON CD
The patent application contains a lengthy table section. A copy of the table is available in electronic form from the USPTO web site (http://seqdata.uspto.gov/?pageRequest=docDetail&DocID=US20080020413A1). An electronic copy of the table will also be available from the USPTO upon request and payment of the fee set forth in 37 CFR 1.19(b)(3).

The Sequence Listing, which is a part of the present disclosure, includes a computer readable form and a written sequence listing comprising nucleotide and/or amino acid sequences of the present invention. The sequence listing information recorded in computer readable form is identical to the written sequence listing. The subject matter of the Sequence Listing is incorporated herein by reference in its entirety.

### INTRODUCTION

The enzyme nicotinamide phosphoribosyltransferase (NMPRTase) has crucial functions in NAD⁺ biosynthesis. This enzyme is involved in several biological phenomena, including longevity and diseases such as cancer and diabetes. While the function of NAD⁺ as a cofactor in oxidation/reduction reactions is well known, NAD⁺ can also be used as a substrate in several biochemical reactions, such as, for example, reactions catalyzed by the NAD⁺-dependent ADP-ribosylating enzyme poly ADP-ribose polymerase (PARP1), sirtuins, and ADP-ribosyl cyclase (Guarente, L., and Picard, F. (2005) Calorie restriction-the SIR2 connection. Cell 120, 473-482.; Marmorstein, R. (2004) Structure and chemistry of the Sir2 family of NAD+-dependent histone/protein deacetylases. Biochem Soc Trans 32, 904-909.; Ziegler, M. (2000) New functions of a long-known molecule. Emerging roles of NAD in cellular signaling. Eur J Biochem 267, 1550-1564). PARP1 is involved in DNA damage and stress responses (Ziegler, 2000). The sirtuins are NAD⁺-dependent histone/protein deacetylases, and are involved in controlling longevity (Guarente and Picard, 2005; Marmorstein, 2004) and neurodegeneration (Araki, T., Sasaki, Y., and Milbrandt, J. (2004) Increased nuclear NAD biosynthesis and SIRT1 activation prevent axonal degeneration. Science 305, 1010-1013). ADP-ribosyl cyclase produces the compound cyclic ADP-ribose from NAD⁺, which is a second messenger that can release calcium ions from their intracellular stores (Guse, A. H. (2005) Second messenger function and the structure-activity relationship of cyclic adenosine diphosphoribose (cADPR). FEBS J 272, 4590-4597; Ziegler, 2000).

A common feature of the biochemical reactions catalyzed by these enzymes is that the glycosidic bond between nicotinamide and ribose in NAD⁺ is broken, destroying the parent NAD⁺ molecule and releasing free nicotinamide (NM). Therefore, these reactions can lead to depletion of the cellular NAD⁺ pool. Nicotinamide phosphoribosyltransferase (NMPRTase) activity is required to replenish the NAD⁺ levels by biosynthesis, salvaging the breakdown product NM and converting it to nicotinamide mononucleotide (NMN, FIG. 1A) (Rongvaux, A., Shea, R. J., Mulks, M. H., Gigot, D., Urbain, J., Leo, O., and Andris, F. (2002) Pre-B-cell colony-enhancing factor, whose expression is up-regulated in activated lymphocytes, is a nicotinamide phorphoribosyltransferase, a cytosolic enzyme involved in NAD biosynthesis. Eur J Immunol 32, 3225-3234). In fact, NMPRTase is believed to be the rate-limiting enzyme for the biosynthesis of NAD⁺ in mouse fibroblasts, and can regulate the function of sirtuins in these cells (Revollo, J. R., Grimm, A. A., and Imai, S.-I. (2004) The NAD biosynthesis pathway mediated by nicotinamide phosphoribosyltransferase regulates Sir2 activity in mammalian cells. J Biol Chem 279, 50754-50763).

There are three pathways of NAD⁺ biosynthesis, each using a different phosphoribosyltransferase (PRTase) to catalyze the formation of nicotinamide mononucleotide (NMN) or nicotinic acid mononucleotide (NAMN) (FIG. 1A). In the de novo biosynthesis pathway, tryptophan is converted to quinolinic acid (QA). In this pathway, quinolinic acid phosphoribosyl transferase (QAPRTase) catalyzes the reaction between QA and phosphoribosylpyrophosphate (PRPP) to form NAMN (FIG. 8). In the salvage pathways, nicotinic acid phosphoribosyl transferase (NAPRTase) catalyzes formation of NAMN from nicotinic acid (NA), while NMPRTase catalyzes the formation NMN from nicotinamide (NM). Despite their functional similarities, the amino acid sequences of these three enzymes are highly divergent (FIG. 1B). A Blast search using the amino acid sequence of NMPRTase cannot find the sequences of the other two PRTases. In addition, NMPRTase is significantly larger (by at least 100 amino acid residues) than the other two PRTases.

The important role of NMPRTase in NAD⁺ biosynthesis has made it an attractive target for the development of novel anti-cancer agents (Hasmann, M., and Schemainda, I. (2003) FK866, a highly specific noncompetitive inhibitor of nicotinamide phosphoribosyltransferase, represents a novel mechanism for induction of tumor cell apoptosis. Cancer Res 63, 7436-7442; Wosikowski, K., Mattern, K., Schemainda, I., Hasmann, M., Rattel, B., and Loser, R. (2002). WK175, a novel antitumor agent, decreases the intracellular nicotinamide adenine dinucleotide concentration and induces the apoptotic cascade in human leukemia cells. Cancer Res 62, 1057-1062). Tumor cells have a high rate of NAD⁺ turnover due to elevated ADP-ribosylation activity, and NMPRTase expression levels are upregulated in some cancers (Hufton, S. E., Moerkerk, P. T., Brandwijk, R., de Bruine, A. P., Arends, J.-W., and Hoogenboom, H. R. (1999). A profile of differentially expressed genes in primary colorectal cancer using suppression substractive hybridization. FEBS Lett 463, 77-82; van Beijnum, J. R., Moerkerk, P. T., Gerbers, A. J., de Bruine, A. P., Arends, J.-W., Hoogenboom, H. R., and Hufton, S. E. (2002). Target validation for genomics using peptide-specific phage antibodies: a study of five gene products overexpressed in colorectal cancer. Int J Cancer 101, 118-127). The compound FK866 is a potent inhibitor of NMPRTase (Kᵢ of 0.3 nM). Application of the FK866 to tumor cells can lead to the depletion of intracellular NAD⁺ levels in tumors, and ultimately induces apoptosis in these cells while having little toxicity to normal cells (Hasmann and Schemainda, 2003; Muruganandham, M., Alfieri, A. A., Matei, C., Chen, Y., Sukenick, G., Schemainda, I., Hasmann, M., Saltz, L. B., and Koutcher, J. A. (2005). Metabolic signatures associated with a NAD synthesis inhibitor-induced tumor apoptosis identified by 1H-decoupled-31P magnetic resonance spectroscopy. Clin Cancer Res 11, 3503-3513; Wosikowski et al., 2002). FK866 also has potent anti-angiogenic effects in a mouse renal cell carcinoma model (Drevs, J., Loser, R., Rattel, B., and Esser, N. (2003). Antiangiogenic potency of FK866/K22.175, a new inhibitor of intracellular NAD biosynthesis, in murine renal cell carcinoma. Anticancer Res 23, 4853-4858).

NMPRTase was originally identified as a secreted growth factor for early B cells, and was named pre-B-cell colony-enhancing factor (PBEF) (Samal, B., Sun, Y., Stearns, G., Xie, C., Suggs, S., and McNiece, I. (1994). Cloning and characterization of the cDNA encoding a novel human pre-B-cell colony-enhancing factor. Mol Cell Biol 14, 1431-1437). It is ubiquitously expressed, with the highest mRNA levels in the liver, bone marrow, and skeletal muscle (Kitani, T., Okuno, S., and Fujisawa, H. (2003) Growth phase-dependent changes in the subcellular localization of pre-B-cell colony-enhancing factor. FEBS Lett 544, 74-78; Samal et al., 1994). More recently, it was found that NMPRTase was secreted by visceral fat tissues also (and named visfatin). NMPRTase may have insulin-mimetic effects (Fukuhara, A., Matsuda, M., Nishizawa, M., Segawa, K., Tanaka, M., Kishimoto, K., Matsuki, Y., Murakami, M., Ichisaka, T., Murakami, H., et al. (2005). Visfatin, a protein secreted by visceral fat that mimics the effects of insulin. Science 307, 426-430), making it a potential target for the development of novel anti-diabetes therapies (Fukuhara et al., 2005; Hug, C., and Lodish, H. F. (2005) Visfatin: a new adipokine. Science 307, 366-367; Sethi, J. K., and Vidal-Puig, A. (2005) Visfatin: the missing link between intra-abdominal obesity and diabetes? Trends Mol Medicine 11, 344-347). Although NMPRTase lacks a secretion signal sequence, it is found in the cytoplasm and nucleus as well as extracellularly (Kitani et al., 2003; Rongvaux, A., Shea, R. J., Mulks, M. H., Gigot, D., Urbain, J., Leo, O., and Andris, F. (2002) Pre-B-cell colony-enhancing factor, whose expression is up-regulated in activated lymphocytes, is a nicotinamide phorphoribosyltransferase, a cytosolic enzyme involved in NAD biosynthesis. Eur J Immunol 32, 3225-3234). In fact, how NMPRTase becomes secreted is currently not known (Hug and Lodish, 2005; Kitani et al., 2003; Rongvaux et al., 2002).

While crystal structure information is now available for QAPRTase and NAPRTase from several bacterial species (Eads, J. C., Ozturk, D., Wexler, T. B., Grubmeyer, C., and Sacchettini, J. C. (1997) A new function for a common fold: the crystal structure of quinolinic acid phosphoribosyltransferase. Structure 5, 47-58; Sharma, V., Grubmeyer, C., and Sacchettini, J. C. (1998) Crystal structure of quinolinic acid phosphoribosyltransferase from Mycobacterium tuberculosis: a potential TB drug target. Structure 6, 1587-1599; Shin, D. H., Oganesyan, N., Jancarik, J., Yokota, H., Kim, R., and Kim, S.-H. (2005) Crystal structure of a nicotinate phosphoribosyltransferase from Thermoplasma acidophilum. J Biol Chem 280, 18326-18335) and yeast (Chappie, J. S., Canaves, J. M., Han, G. W., Rife, C. L., Xu, Q., and Stevens, R. C. (2005) The structure of a eukaryotic nicotinic acid phosphoribosyltransferase reveals structural heterogeneity among type II PRTases. Structure 13, 1385-1396) no structures are currently available for any NMPRTase.

### SUMMARY

The present inventors have developed, in various aspects of the present teachings, crystals of nicotinamide phosphoribosyltransferase (NMPRTase). In some aspects, the crystals comprise not only NMPRTase, but also at least one NMPRTase ligand. In some aspects, the crystal can comprise a complex of NMPRTase and a substrate or reaction product of a biochemical reaction catalyzed by NMPRTase, such as nicotinamide mononucleotide (NMN). In other aspects, a crystal can comprise a complex of NMPRTase and an NMPRTase inhibitor, such as (E)-N-[4-(1-benzoylpiperidin-4-yl)butyl]-3-(pyridin-3-yl)acrylamide (FK866). In various aspects, the NMPRTase can be a human NMPRTase or a murine NMPRTase, comprising sequences set forth in SEQ ID NO: 1 and SEQ ID NO: 2, respectively. An NMPRTase protein of the present teachings can further comprise a carboxyl terminal histidine sequence. In yet other aspects, the sequence of an NMPRTase can comprise addition or substitutions of single amino acids, such as substitution of from one up to about 5 amino acids with methionines.

In various configurations, graphical depictions of three-dimensional structures of NMPRTases, with or without ligands, are also provided. In various aspects, these images are three-dimensional images provided on a digital computer using atomic coordinate data determined using x-ray crystallographic analysis of the NMPRTase crystals.

The present teachings also provide methods of forming NMPRTase crystals. These methods comprise expressing an NMPRTase in cells such as E. coli cells; purifying the expressed NMPRTase, and subjecting the purified NMPRTase to crystallizing conditions.

The present teachings also provide methods of identifying a compound that modifies nicotinamide phosphoribosyltransferase (NMPRTase) activity. In various aspects, these methods comprise (A) designing a candidate compound predicted to interact with an NMPRTase (for example through hydrogen-bonding, van der Waals forces, pi-stacking, or the formation of at least one bond, such as an ionic and/or covalent bond) wherein the designing comprises computer-aided design using atomic coordinates of an NMPRTase or an NMPRTase-ligand complex; (B) obtaining the candidate compound; (C) contacting an NMPRTase with the candidate compound in vitro; and (D) detecting inhibition or enhancement of NMPRTase activity. In various configurations, the atomic coordinates can be that of an NMPRTase without a ligand, or with one or more ligands such as a substrate, a product of a reaction catalyzed by NMPRTase, or an inhibitor.

The present teachings also provide compositions and methods for treating cancer. In various aspects of these teachings, a composition for use in treating cancer can comprise an NMPRTase inhibitor identified by the methods described herein. These methods comprise administering to a patient in need of treatment, a composition comprising an NMPRTase inhibitor identified using the methods described herein.

The present teachings also provide compositions and methods for treating diabetes. In various aspects of these teachings, a composition for use in treating diabetes can comprise an NMPRTase, NMPRTase analog, and/or effector of NMPRTase activity identified by the methods described herein. These methods comprise administering to a patient in need of treatment, a composition comprising NMPRTase, NMPRTase analog, and/or effector of NMPRTase activity identified using the methods described herein.

Some embodiments of the present teachings include computer-readable media encoded with one or more sets of three dimensional coordinates of one or more three dimensional structures of NMPRTase. In these embodiments, a structure can substantially conform to the three dimensional coordinates represented in a table comprising atomic coordinates of an NMPRTase as determined by x-ray crystallography. In these aspects, using a graphical display software program, the set of three dimensional coordinates can be used to create an electronic file which can be visualized on a computer capable of representing the electronic file as one or more three dimensional images.

Embodiments of the present teachings include methods for designing drugs which interfere with the activity of an NMPRTase. These methods comprise (a) providing on a digital computer a three-dimensional structure of a NMPRTase; (b) using software comprised by the digital computer to design a chemical compound which is predicted to bind to the NMPRTase; (c) obtaining the chemical compound; and (d) evaluating the chemical compound for an ability to interfere with an activity of the NMPRTase.

Similarly, certain embodiments of the present teachings include methods for designing drugs which enhance activity of an NMPRTase. These methods comprise (a) providing on a digital computer a three-dimensional structure of a NMPRTase; (b) using software comprised by the digital computer to design a chemical compound which is predicted to bind to the NMPRTase; (c) obtaining the chemical compound; and (d) evaluating the chemical compound for an ability to enhance NMPRTase activity.

Various configurations of the present teachings include methods for generating a model of a three dimensional structure of NMPRTase. These methods comprise (a) providing an amino acid sequence of a known NMPRTase and an amino acid sequence of a target NMPRTase; (b) identifying structurally conserved regions shared between the known NMPRTase and the target NMPRTase; and (c) assigning atomic coordinates from the conserved regions to the target NMPRTase.

In similar aspects, the present teachings include methods for determining the three dimensional structure of a target NMPRTase. These method comprise (a) providing an amino acid sequence of a target NMPRTase, wherein the three dimensional structure of the target NMPRTase is not known; (b) predicting the pattern of folding of the amino acid sequence in a three dimensional conformation using a fold recognition algorithm; and (c) comparing the pattern of folding of the target structure amino acid sequence with the three dimensional structure of a known NMPRTase.

### BRIEF DESCRIPTION OF THE DRAWINGS

FIG. 1. illustrates sequence alignment of NMPRTase with other phosphoribosyltransferases involved in NAD+ biosynthesis. (A). Chemical structures of nicotinamide (NM), nicotinic acid (NA), quinolinic acid (QA), nicotinamide mononucleotide (NMN), and nicotinic acid mononucleotide (NAMN). (B). Structure-based sequence alignment of human NMPRTase (hsNMPRT) (SEQ. ID No. 1) with NAPRTase from T. acidophilum (taNAPRT) (SEQ. ID No. 3) (Shin et al., 2005) and QAPRTase from M. tuberculosis (mtQAPRTase) (SEQ. ID No.4) (Sharma et al., 1998). Residues in taNAPRT and mtQAPRT that are within 3 Å of the equivalent Ca position in NMPRTase are shown in blue. The secondary structure elements in the NMPRTase structure are labeled (S.S.). Residues shown in magenta are in the dimer interface of NMPRTase. A dot represents a deletion.

FIG. 2 illustrates structure of the human NMPRTase monomer. (A). Stereo diagram showing a schematic representation of the structure of NMPRTase in complex with NMN. The b-strands, a-helices and the three domains of the protein are labeled. NMN is shown in green for carbon atoms. The bound position of FK866 is also shown (in black). (B). Structure of taNAPRTase in complex with NAMN (Shin et al., 2005). (C). Structure of mtQAPRTase in complex with NAMN (Sharma et al., 1998). Produced with Ribbons (Carson, M. (1987) Ribbon models of macromolecules. J Mol Graphics 5, 103-106). See FIG. 10 for stereo versions of panels B and C.

FIG. 3 illustrates structure of the human NMPRTase dimer. (A). Stereo diagram showing the dimer of NMPRTase in complex with NMN. One monomer of the dimer is shown in cyan, and other monomer in yellow. NMN is shown in green for carbon atoms, and FK866 in black. (B). Structure of taNAPRTase dimer in complex with NAMN (Shin et al., 2005). (C). Structure of mtQAPRTase dimer in complex with NAMN (Sharma et al., 1998). Produced with Ribbons (Carson, 1987). See FIG. 11 for stereo versions of panels B and C.

FIG. 4 illustrates binding mode of NMN and the active site of NMPRTase. (A). Final 2Fo-Fc electron density map for NMN at 2.2 Å resolution. The contour level is at 1 s. Produced with Setor (Evans, S. V. (1993). SETOR: hardware lighted three-dimensional solid model representations of macromolecules. J Mol Graphics 11, 134-138). (B). Stereo diagram showing the NMN binding site of NMPRTase. The two monomers are colored cyan and yellow, and their side chains in gray and magenta, respectively. NMN is shown in green, and two phosphate groups in the binding site are labeled. (C). Comparison of the binding site of NMN in NMPRTase with that of NAMN in taNAPRTase (Shin et al., 2005). The taNAPRTase structure is shown in gray, and NAMN is in magenta. The red arrow indicates the shift in the position of strand b8 between the structures of taNAPRTase and NMPRTase. Panels B and C produced with Ribbons (Carson, 1987).

FIG. 5 illustrates a plot of the maximal velocity of NMPRTase as a function of FK866 concentration.

FIG. 6 illustrates the FK866 binding site of human NMPRTase. (A). Final 2Fo-Fc electron density map for FK866 at 2.1 Å resolution. The contour level is at 1 s. Produced with Setor (Evans, 1993). (B). Stereo diagram showing the FK866 binding site of NMPRTase. The two monomers are colored cyan and yellow, and their side chains in green and magenta, respectively. FK866 is shown in black, and a water molecule is shown as a sphere in red. Produced with Ribbons (Carson, 1987). (C). Molecular surface of NMPRTase in the region of the FK866 binding site. The compound is located in a tunnel in the NMPRTase dimer. Produced with Grasp (Nicholls, A., Sharp, K. A., and Honig, B. (1991) Protein folding and association: insights from the interfacial and thermodynamic properties of hydrocarbons. Proteins 11, 281-296). (D). Structural comparison between NMPRTase and taNAPRTase in the FK866 binding site. The taNAPRTase structure is shown in gray. Produced with Ribbons (Carson, 1987).

FIG. 7 illustrates a comparison of the structure of human NMPRTase (in green) in complex with FK866 (black) and the structure of the free enzyme of murine NMPRTase (yellow).

FIG. 8 illustrates pathways for NAD+ biosynthesis. The de novo biosynthetic pathway utilizes Trp as the precursor, while the salvage pathways use NM or NA as the precursor. Recently, a fourth pathway was identified, using nicotinamide riboside (NR) as the precursor.

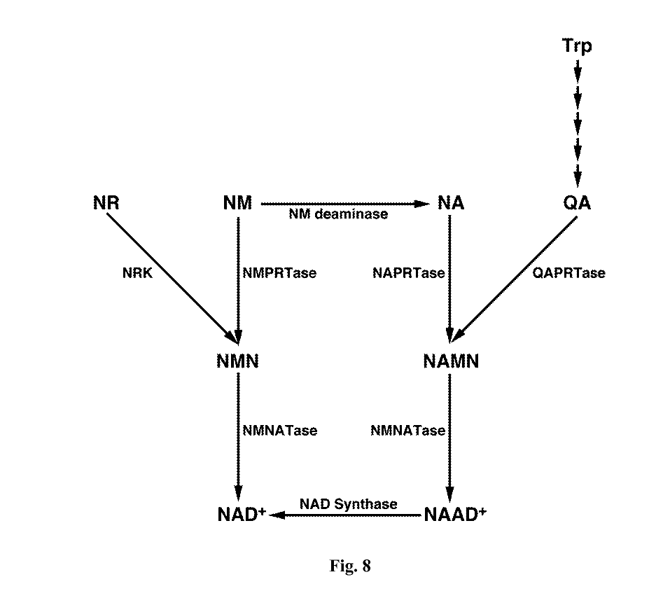

FIG. 9 illustrates overlap of the bound positions of NMN (green) and FK866 (black) to NMPRTase.

FIG. 10 illustrates a stereo diagram showing the structure of taNAPRTase monomer in complex with NAMN (top) and the structure of mtQAPRTase monomer in complex with NAMN (bottom).

FIG. 11 illustrates a stereo diagram showing the dimer of taNAPRTase in complex with NAMN (top) and of mtQAPRTase in complex with NAMN (bottom).

FIG. 12 illustrates a comparison of the structure of human NMPRTase (in cyan) in complex with NMN (green) and the structure of the free enzyme of murine NMPRTase (gray).

FIG. 13 illustrates catalytic activity of NMPRTase as a function of substrate (NM) concentration.

### DETAILED DESCRIPTION

The present invention relates to the discovery of the three-dimensional structure of nicotinamide phosphoribosyltransferases (NMPRTases), NMPRTase-ligand Complexes, models of such three-dimensional structures, a method of structure-based drug design using such structures, the compounds identified by such methods and the use of such compounds in therapeutic compositions.

Some aspects of the present teachings include models of NMPRTase and NMPRTase-Ligand Complexes in which each model represents a three dimensional structure of an NMPRTase or NMPRTase-Ligand Complex. Other aspects of the present teachings include the three dimensional structures of NMPRTase and NMPRTase-Ligand Complexes. A three dimensional structure of human NMPRTase-Ligand Complexes substantially conform with the atomic coordinates represented in Table 1 and Table 2, wherein Table 1 provides the atomic coordinates of human NMPRTase in a complex with nicotinamide mononucleotide (NMN), and Table 2 provides the atomic coordinates of human NMPRTase in a complex with the NMPRTase inhibitor (E)-N-[4-(1-benzoylpiperidin-4-yl) butyl]-3-(pyridin-3-yl) acrylamide (FK866). In addition, Table 3 provides the atomic coordinates of murine NMPRTase free enzyme. According to the present invention, the use of the term “substantially conforms” refers to at least a portion of a three dimensional structure of an NMPRTase or an NMPRTase-Ligand Complex which is sufficiently spatially similar to at least a portion of a specified three dimensional configuration of a particular set of atomic coordinates (e.g., those represented in Tables 1, 2, or 3) to allow the three dimensional structure of an NMPRTase or an NMPRTase-Ligand Complex to be modeled or calculated using a particular set of atomic coordinates as a basis for determining the atomic coordinates defining the three dimensional configuration of an NMPRTase or an NMPRTase-Ligand Complex.

Accordingly, in various aspects, the present teachings provide crystals comprising a complex, wherein the complex comprises a nicotinamide phosphoribosyltransferase (NMPRTase) and at least one ligand of NMPRTase. In some configurations, a crystal can comprise human NMPRTase such as a human NMPRTase comprising an amino acid sequence as set forth in SEQ ID NO: 1. The human NMPRTase, in some aspects, can further comprise a histidine tag at its carboxyl terminal, such as a hexahistidine tag. In some aspects, a ligand can be a reaction substrate or a reaction product, such as, for example, nicotinamide mononucleotide (NMN). A crystal of such a complex can belong to to space group C2. In addition, in some aspects, unit cells of such crystals can have cell parameters of a=253.07 Å, b=101.37 Å, c=148.20 Å, and β=125.48°. Furthermore, the NMPRTase-NMN crystal can be composed of asymetric units, each containing six copies of the NMPRTase-NMN complex. For example, the six copies can include two dimers and two monomers at the crystallographic two-fold axis. Accordingly, in some aspects the present teachings provide a three dimensional computer image of the three dimensional structure of the crystal. This three dimensional structure can substantially conform with the three dimensional atomic coordinates set forth in Table 1.

In some configurations, a crystal of an NMPRTase-NMN complex can further comprise a first free phosphate group hydrogen bonded to the side chains of Arg196, His247, Arg311 and Tyr18′ of the NMPRTase, and a second free phosphate hydrogen bonded to to the 2′-hydroxyl of the ribose of the NMN. In some aspects, a crystal comprising a complex of NMPRTase and NMN can have a structure in which the nicotinamide ring of NMN is sandwiched between the sidechains of Phe193 and Tyr18′, wherein pi-stacking interactions occur.

In various aspects, a crystal of the present teachings can be a crystal comprising NMPRTase and an NMPRTase inhibitor. In some configurations, the NMPRTase can be human NMPRTase having the sequence set forth in SEQ ID NO: 1, and can also have a histidine tag at its carboxyl terminus, as described above. In various aspects, the inhibitor can (E)-N-[4-(1-benzoylpiperidin-4-yl) butyl]-3-(pyridin-3-yl) acrylamide (FK866). In these aspects, a crystal comprising an NMPRTase-FK866 complex can belong to space group P21. In addition, a unit cell of the crystal can have cell parameters of a=60.78 Å, b=105.89 Å, c=83.43 Å, and β=96.45°. Furthermore, the NMPRTase-FK866 crystal can be composed of asymetric units, each containing two copies of the NMPRTase-NMN complex. In some configurations, a crystal of an NMPRTase-ligand complex can be of sufficient purity to determine atomic coordinates of the NMPRTase protein by X-ray diffraction to a resolution as low as about 2.1 Å.

In some aspects, a crystal comprising a complex of NMPRTase and an NMPRTase inhibitor such as FK866 can have a structure in which the inhibitor is hydrogen-bonded to the side chain hydroxyl of Ser275 of the NMPRTase. In further aspects, a crystal comprising a complex of NMPRTase and an NMPRTase inhibitor, such as FK866, can have a structure in which an aromatic ring of the inhibitor, for example the pyridyl ring of FK866, is sandwiched between the sidechains of Phe193 and Tyr18′, wherein pi-stacking interactions occur. In yet other aspects, the crystal can further comprise a water molecule, wherein the water molecule is hydrogen bonded to the amide nitrogen of an NMPRTase inhibitor such as FK866 and to the side chains of Asp219 and Ser241 of the NMPRTase. In still further aspects, the crystal structure comprises various combinations of the above features.

In yet other aspects, the present teaching provides for these crystals. A three dimensional computer image of the three dimensional structure of the NMPRTase-inhibitor complex comprising a crystal. In these aspects, a structure can substantially conform with the three dimensional coordinates listed in Table 2.

In some aspects, the present teachings include a crystal comprising a substantially pure murine nicotinamide phosphoribosyltransferase (NMPRTase). In some aspects, the crystal can be a crystal of the free enzyme, i.e., without a ligand. The murine NPRTase can comprise the sequence set forth in SEQ ID NO: 2, and furthermore can also have a histidine tag, such as a hexhistidine tag, attached to its carboxyterminal.

In various configurations, a crystal of these aspects can comprise unit cells having cell parameters of a=60.26 Å, b=107.73 Å, c=83.28 Å, and β=96.56°. Furthermore, the crystals can be substantially isomorphous to crystals of a human NMPRTase:FK866 complex. In various configurations, a crystal of these aspects can be sufficiently pure to determine atomic coordinates of the NMPRTase protein by X-ray diffraction to a resolution of about 2.1 Å.

In yet other aspects, the present teaching provides for these crystals. A three dimensional computer image of the three dimensional structure of the NMPRTase-inhibitor complex comprising a crystal. In these aspects, a structure can substantially conform with the three dimensional coordinates listed in Table 3.

In various configurations, a crystal of the present teachings can comprise a substantially pure human nicotinamide phosphoribosyltransferase (NMPRTase). In some aspects, a crystal of these configurations can be sufficiently pure for determining atomic coordinates of the NMPRTase by X-ray diffraction to a resolution of about 2.7 Å. In some configurations, the NMPRTase can have the sequence set forth in SEQ ID NO: 1, and in related aspects, the NMPRTase can further comprise a histidine tag such as a hexahistidine tag attached to its carboxyl terminus. In yet other configurations, The human NMPRTase can comprise an F132M/I151M double mutant of human NMPRTase The crystal can belong to space group P212121 and can comprise unit cells having parameters a=87.98 Å, b=93.43 Å, and c=244.26 Å. Furthermore, the NMPRTase crystal can be composed of asymetric units, each containing four copies of the NMPRTase.

In some configurations of the present teachings, methods are provided for forming nicotinamide phosphoribosyltransferase (NMPRTase) crystals. In various aspects, these methods comprise (a) expressing an NMPRTase in cells; (b) purifying the NMPRTase expressed in (a); and (c) subjecting the NMPRTase purified in (b) to crystallizing conditions. Crystallizing conditions can be conditions well known to skilled artisans. In various aspects, the NMPRTase can be a human or a murine NMPRTase, including an F132M/I151M double mutant of a human NMPRTase. In some configurations, the cells expressing the NMPRTase can be prokaryotic cells such as E. coli cells, and the NMPRTase can include a sequence such as set forth in SEQ ID NO: 1 or SEQ ID NO: 2, and can, in some aspects, further comprise a histidine tag at the protein's carboxyl terminus, such as a hexahistidine tag. In some aspects, cells expressing an NMPRTase such as E. coli cells can comprise a nucleic acid sequence encoding an NMPRTase, such as a DNA sequence encoding an NMPRTase. The nucleic acid sequence can be operably linked to a promoter such as a heterologous promoter. Furthermore, in some aspects, the nucleic acid linked to a promoter can be comprised by a vector such as, for example, a plasmid or viral vector such as a bacteriophage vector. The promoter can be, in some configurations, an inducible promoter such as a lac promoter or a bacteriophage promoter that, upon induction, causes the cell to express high levels of the NMPRTase. In some aspects it is desirable to produce chemically labeled NMPRTase. For example, where seleno-methionine labeled NMPRTase is desired, cells such as, for example, B834(DE3) E. coli cells (Novagen) expressing NMPRTase can be grown in a medium such as defined LeMaster medium supplemented with seleno-methionine (Hendrickson, W. A., Horton, J. R., and LeMaster, D. M. (1990) Selenomethionyl proteins produced for analysis by multiwavelength anomalous diffraction (MAD): a vehicle for direct determination of three-dimensional structure. EMBO J 9,1665-1672).

In these aspects, purification of an NMPRTase of these configurations can comprise subjecting a lysate from cells expressing the NMPRTase, to purification procedures well known to skilled artisans such as procedures described in standard references such as Sambrook, J., et al., Molecular Cloning: A Laboratory Manual, 3rd ed. Cold Spring Harbor Laboratory Press, Cold Spring Harbor, N.Y., 2001. Accordingly, in some aspects, NMPRTase purification can comprise subjecting a cell lysate to procedures such as nickel-agarose chromatography, anion exchange chromatography and gel filtration chromatography. Furthermore, in some aspects, following purification, NMPRTase can be incubated with at least one NMPRTase ligand such as NMN and/or FK866 prior to crystallization.

In various aspects, crystallization of NMPRTase can be effected using methods adapted from those known to skilled artisans, such as, for example, subjecting chromatographically purified NMPRTase to sitting-drop vapor diffusion.

In some configurations of the present teachings, methods are disclosed for identifying compounds which modify nicotinamide phosphoribosyltransferase (NMPRTase) activity. In various aspects, these methods comprise (A) designing a candidate compound predicted to form at least one bond with an NMPRTase, wherein the designing comprises computer-aided design using atomic coordinates of an NMPRTase or an NMPRTase-ligand complex; (B) obtaining the candidate compound; (C) contacting an NMPRTase with the candidate compound in vitro; and (D) detecting inhibition or enhancement of NMPRTase activity. In various aspects, the atomic coordinates can be those set forth in Table 1, Table 2 and/or Table 3.

Examples of inhibitory compounds of the present teachings are compounds that interact directly with an NMPRTase protein, and can, for example, inhibit binding between an NMPRTase and an NMPRTase substrate. According to the present teachings, examples of suitable therapeutic compounds include peptides or other organic molecules. Suitable organic molecules include small organic molecules, of a molecular weight of at least about 80 up to about 2,000 daltons. Therapeutic compounds of the present teachings can be designed using structure based drug design. Structure based drug design refers to the use of computer simulation to predict a conformation of a peptide, polypeptide, protein, or conformational interaction between a peptide or polypeptide, and a therapeutic compound. In the present teachings, knowledge of the three dimensional structure of the NMPRTase and its binding sites for both NMN and the FK866 provide one of skill in the art the ability to design a therapeutic compound that binds to NMPRTase, is stable and results in enhancement or inhibition of NMPRTase biochemical activity. For example, knowledge of the three dimensional structure of the NMN binding site provides to a skilled artisan the ability to design an analog of NMN which can function as a competitive inhibitor of NMPRTase.

Suitable structures and models useful for structure-based drug design are disclosed herein. Models of target structures to use in a method of structure-based drug design include models produced by any modeling method disclosed herein, such as, for example, molecular replacement and fold recognition related methods. In some aspects of the present teachings, structure based drug design can be applied to a structure of NMPRTase in complex with FK866 or NMN.

One embodiment of the present teachings is a method for designing a drug which interferes with an activity of an NMPRTase. In various configurations, the method comprises providing a three-dimensional structure of an NMPRTase-Ligand Complex; and designing a chemical compound which is predicted to bind to the NMPRTase. The designing can comprise using physical models, such as, for example, ball-and-stick representations of atoms and bonds, or on a digital computer equipped with molecular modeling software. In some configurations, these methods can further include synthesizing the chemical compound, and evaluating the chemical compound for ability to interfere with NMPRTase activity.

According to the present teachings, designing a compound can include creating a new chemical compound or searching databases of libraries of known compounds (e.g., a compound listed in a computational screening database containing three dimensional structures of known compounds). Designing can also include simulating chemical compounds having substitute moieties at certain structural features. In some configurations, designing can include selecting a chemical compound based on a known function of the compound. In some configurations designing can comprise computational screening of one or more databases of compounds in which three dimensional structures of the compounds are known. In these configurations, a candidate compound can be interacted virtually (e.g., docked, aligned, matched, interfaced) with the three dimensional structure of NMPRTase or an NMPRTase-Ligand Complex by computer equipped with software such as, for example, the AutoDock software package, (The Scripps Research Institute, La Jolla, Calif.) or software described by Humblet and Dunbar, Animal Reports in Medicinal Chemistry, vol. 28, pp. 275-283, 1993, M Venuti, ed., Academic Press. Methods for synthesizing candidate chemical compounds are known to those of skill in the art.

Various other methods of structure-based drug design are disclosed in references such as Maulik et al., 1997, Molecular Biotechnology: Therapeutic Applications and Strategies, Wiley-Liss, Inc., which is incorporated herein by reference in its entirety. Maulik et al. disclose, for example, methods of directed design, in which the user directs the process of creating novel molecules from a fragment library of appropriately selected fragments; random design, in which the user uses a genetic or other algorithm to randomly mutate fragments and their combinations while simultaneously applying a selection criterion to evaluate the fitness of candidate ligands; and a grid-based approach in which the user calculates the interaction energy between three dimensional structures and small fragment probes, followed by linking together of favorable probe sites.

In some configurations, a chemical compound that binds to the active site or to a non-competitive inhibitor binding site of NMPRTase can associate with an affinity of at least about 10-6 M, at least about 10-7 M, or at least about 10-8 M.

In some configurations, a candidate compound predicted to form at least one bond with an NMPRTase can be a candidate compound predicted to form at least one bond with an NMPRTase substrate binding site. Furthermore, a substrate binding site can comprise at least one atom of an NMPRTase active site. In some aspects, a substrate binding site can be located adjacent a substrate-binding pocket, i.e., a space defined by amino acid residues which have at least one atom situated 10 Å or less from a substrate such as NMN bound to the active site. Accordingly, in some configurations, amino acids defining a substrate-binding pocket, including a substrate binding site, can comprise, in a first monomer of a human NMPRTase dimer, His191, Asp192, Phe193, Gly194, Tyr195, Arg196, Gly 197, Val 198, Ser 199, Gly217, Thr218, Asp219, Thr 220, Val 221, Tyr 240, Ser241, Val242, Pro243, Ala244, Ala245, Glu246, His247, Ser248, Val274, Ser275, Val276, Val277, Ser278, Asp279, Ile309, Ile 310, Arg 311, Pro312, Asp313, Ser314, Gly315, Pro317, Ile351, Gln352, Gly353, Asp354, Gly 355, Val356, Asp357, Thr360, Phe380, Gly381, Ser382, Gly383, Gly384, Gly 385, Leu386, Leu387, Gln388 and Lys 389, and in a second monomer of the dimer, Thr15, Asp16, Ser17, Tyr18, Lys 19, Val 20, Thr 21, His 22, Gln 25, Arg 40, His90, Phe91, Glu149, Thr150, Val153, Trp156, Le 390, Thr391, Arg392, Asp393, Leu394, Asn396, Cys397, Ser398, Phe399, Lys400, Lys415, Pro417, and Lys 423.

In some aspects, a candidate compound can be a compound predicted to be an NMPRTase competitive inhibitor such as a substrate analog, a non-competitive inhibitor, or an uncompetitive inhibitor, defined as an inhibitor which is incapable of binding to free enzyme, but can bind an enzyme-substrate complex. In some configuration, a compound predicted to form at least one bond with an NMPRTase can be a compound predicted to form at least one bond with an amino acid comprising an inhibitor binding site of an NMPRTase, such as, for example, the binding site of (E)-N-[4-(1-benzoylpiperidin-4-yl) butyl]-3-(pyridin-3-yl) acrylamide (FK866). In some aspects, a inhibitor-binding site can be located adjacent a inhibitor-binding pocket, i.e., a space defined by amino acid residues which have at least one atom situated 10 Å or less from an inhibitor such as FK866 bound to the enzyme. Accordingly, in some configurations, amino acids defining an inhibitor-binding pocket can include, in a first monomer of a human NMPRTase dimer, Thr 15, Asp 16, Ser 17, Tyr18, Lys19, Val 20, Thr21, His 22, Gln 25, His 90, Phe 91, Asn146, Glu149, Thr150, Val153, Arg 392, Phe399 and Lys415, and in the second monomer of the dimer, Leu172, Leu176, Leu183, Asp184, Gly185, Leu186, Glu187, Tyr 188, Lys189, Leu190, His191, Asp192, Phe193, Gly194, Tyr195, Arg196, Gly197, Phe215, Lys216, Gly217, Thr218, Asp219, Thr220, Val221, Gly239, Tyr240, Ser241, Val 242, Pro243, Ala244, Ala245, Glu246, His247, Val272, Pro273, Val274, Ser275, Val276, Val277, Ser 278, Arg302, Ser303, Thr304, Gln305, Ala306, Pro307, Leu308, Ile309, Ile310, Arg311, Pro312, Arg313, Leu325, Leu343, Leu344, Pro345, Pro346, Tyr347, Leu348, Arg349, Val350, Ile351, Gln352, Gly353, Asp354, Met368, Ser374, Ile375, Glu376, Asn377, Ile378, Ala379, Phe380, Gly381, Ser382, Gly 383 and Gly384. In yet other aspects, an inhibitor-binding pocket can comprise an inhibitor-binding domain, a space defined by amino acids which have at least one atom situated 5 Å or less from an inhibitor such as FK866 bound to the enzyme. Accordingly, amino acids defining an inhibitor-binding domain can include, in a first monomer of a human NMPRTase dimer, Asp16 and Tyr18, and in a second monomer of the dimer, Tyr188, Lys189, His191, Phe193, Arg196, Asp219, Val242, Pro243, Ala244, Pro273, Ser275, Pro 307, Ile309, Arg311, Arg349, Val350, Ile351, Glu376, Asn377, Ile378 and Ala379.

Accordingly, candidate compound predicted to form at least one bond with an NMPRTase can be, in various aspects, an antibody, a peptide, an aptamer, an avimer (Jeong, K. J., et al., Nature Biotechnology 23: 1493-1494, 2005), and an organic molecule having a molecular weight of at least about 80 daltons up to about 2000 daltons. In some configurations, a candidate compound can be a candidate NMPRTase inhibitor, while in other configurations, a candidate compound can be a candidate NMPRTase activity enhancer. In some aspects, obtaining the candidate compound can comprise synthesizing the candidate compound. Synthetic methods well known to skilled artisans can be used to synthesize candidate compounds. Guidance for organic synthesis can be found in in standard organic chemistry texts, such as, for example, Smith, M. B. and March, J., March's Advanced Organic Chemistry: Reactions, Mechanisms and Structure—5th edition. New York: John Wiley & Sons, 2001.

In various aspects, the present teachings disclose compositions for treating a cancer. In various configurations, a composition can comprise an inhibitor of an NMPRTase identified by a method described above. Furthermore, a composition can also comprise an anticancer chemotherapeutic. In non-limiting example, the inhibitor can be an antibody, an avimer, or an organic molecule having a molecular weight of at least about 80 daltons up to about 2000 daltons. An antibody can be, for example, directed against the active site of NMPRTase as described by atomic coordinates. In various aspects, an anticancer chemotherapeutic can be, in non-limiting example, colchicine, doxorubicin, adriamycin, vinblastine, digoxin, saquinivir or paclitaxel. In some embodiments, a composition can comprise an NMPRTase covalently attached to an anticancer chemotherapeutic, wherein the inhibitor is an inhibitor identified in accordance with teachings herein.

In related aspects, the present teachings contemplate methods of treating a cancer. In various configurations, these methods comprise administering to a patient in need of treatment, a composition comprising an inhibitor of an NMPRTase inhibitor identified in accordance with the methods described herein. In some configurations, the methods can comprise administering to a patient in need of treatment an NMPRTase inhibitor covalently attached to an anticancer chemotherapeutic, wherein the inhibitor is one identified using the methods described herein.

In various aspects, the present teachings disclose compositions for treating diabetes. Such compositions can comprise NMPRTase, NMPRTase analogs, and/or effectors of NMPRTase activity identified in accordance with the methods disclosed herein, and optionally insulin. Such effectors can include NMPRTase enhancers or NMPRTase inhibitors, depending upon the desired regulatory activity.

The present teachings also contemplate, in some embodiments, methods of treating diabetes. In various aspects, these methods comprise administering to a patient in need of treatment, a composition comprising NMPRTase, NMPRTase analogs, and/or effectors of NMPRTase activity identified in accordance with the methods described herein. In some configurations, a composition can further comprise insulin, and in other configurations, the insulin can be covalently attached.

In various aspects, the present teachings also include three dimensional computer images of a three dimensional structure of a nicotinamide mononucleotide phosphoribosyltransferase, wherein the structure substantially conforms with the three dimensional atomic coordinates determined from a crystal of either an NMPRTase free enzyme, or an NMPRTase in complex with a ligand such as an reaction product or an inhibitor. In non-limiting example, the three dimensional coordinates can be coordinates comprised by a table such as Table 1, Table 2, Table 3 or a combination thereof.

In various aspects, the present teachings also contemplate a computer-readable medium encoded with a set of three dimensional coordinates of an NMPRTase as represented in a table, wherein, using a graphical display software program, the three dimensional coordinates create an electronic file that can be visualized on a computer capable of representing the electronic file as a three dimensional image. A table of these aspects can be, in non-limiting examples, Table 1, Table 2, Table 3 or a combination thereof.

In related aspects, the present teachings also contemplate a computer-readable medium encoded with one or more sets of three dimensional coordinates of one or more three dimensional structures wherein each structure substantially conforms to the three dimensional coordinates represented in a table selected from Table 1, Table 2, Table 3 and a combination thereof, wherein, using a graphical display software program, the set of three dimensional coordinates create an electronic file that can be visualized on a computer capable of representing said electronic file as one or more three dimensional images.

In some embodiments, the present teachings disclose methods for designing a drug which interferes with the activity of a nicotinamide phosphoribosyltransferase (NMPRTase). These methods comprise (a) providing on a digital computer a three-dimensional structure of a NMPRTase; (b) using software comprised by the digital computer to design a chemical compound which is predicted to bind to the NMPRTase; (c) obtaining the chemical compound; and (d) evaluating the chemical compound for an ability to interfere with an activity of the NMPRTase. In some configurations, a chemical compound of these methods can be designed by computational interaction with reference to a site of a three-dimensional structure of an NMPRTase or an NMPRTase-ligand complex, wherein three-dimensional structure comprises atomic coordinates that substantially conform to atomic coordinates set forth in a table such as Table 1, Table 2, Table 3 or a combination thereof. In certain aspects, obtaining the chemical compound can comprise synthesizing the candidate compound.

In some embodiments, the present teachings disclose methods for designing a drug which enhances activity of a nicotinamide phosphoribosyltransferase (NMPRTase). These methods comprise (a) providing on a digital computer a three-dimensional structure of a NMPRTase; (b) using software comprised by the digital computer to design a chemical compound which is predicted to bind to the NMPRTase; (c) obtaining the chemical compound; and (d) evaluating the chemical compound for an ability to enhance NMPRTase activity. In some configurations, a chemical compound of these methods can be designed by computational interaction with reference to a site of a three-dimensional structure of an NMPRTase or an NMPRTase-ligand complex, wherein three-dimensional structure comprises atomic coordinates that substantially conform to atomic coordinates set forth in a table such as Table 1, Table 2, Table 3 or a combination thereof. In certain aspects, obtaining the chemical compound can comprise synthesizing the candidate compound.

In various aspects, the present teachings disclose methods for generating a model of a three dimensional structure of NMPRTase. These methods comprise (a) providing an amino acid sequence of a known NMPRTase and an amino acid sequence of a target NMPRTase; (b) identifying structurally conserved regions shared between the known NMPRTase and the target NMPRTase; and (c) assigning atomic coordinates from the conserved regions to the target NMPRTase. In various aspects, the known NMPRTase can have a three dimensional structure described by atomic coordinates that substantially conform to atomic coordinates set forth in a table such as Table 1, Table 2, Table 3 or a combination thereof.

In various embodiments, the present teachings disclose methods for determining a three dimensional structure of a target NMPRTase. These methods comprise (a) providing an amino acid sequence of a target NMPRTase, wherein the three dimensional structure of the target NMPRTase is not known; (b) predicting the pattern of folding of the amino acid sequence in a three dimensional conformation using a fold recognition algorithm; and (c) comparing the pattern of folding of the target structure amino acid sequence with the three dimensional structure of a known NMPRTase. In various aspects, the known NMPRTase can have a sequence set forth in SEQ ID NO: 1 or SEQ ID NO: 2. Furthermore, in some configurations, the three dimensional structure of a known NMPRTase can be described by atomic coordinates that substantially conform to atomic coordinates set forth in a table, such as Table 1, Table 2, Table 3 or a combination thereof.

In various aspects, the design of a chemical compound possessing stereochemical complementarity can be accomplished by means of techniques that optimize, chemically or geometrically, the “fit” between a chemical compound and a target site. Such techniques are disclosed by, for example, Sheridan and Venkataraghavan, Acc. Chem Res., vol. 20, p. 322, 1987: Goodford, J. Med. Chem., vol. 27, p. 557, 1984; Beddell, Chem. Soc. Reviews, vol. 279, 1985; Hol, Angew. Chem., vol. 25, p. 767, 1986; and Verlinde and Hol, Structure, vol. 2, p. 577, 1994, each of which are incorporated by this reference herein in their entirety.

Some embodiments of the present invention for structure-based drug design comprise methods of identifying a chemical compound that complements the shape of an NMPRTase active site or NMPRTase inhibitor binding site. Such methods are referred to herein as a “geometric approach.” In a geometric approach of the present invention, the number of internal degrees of freedom (and the corresponding local minima in the molecular conformation space) can be reduced by considering only the geometric (hard-sphere) interactions of two rigid bodies, where one body (the active site) contains “pockets” or “grooves” that form binding sites for the second body (the complementing molecule, such as a ligand). The geometric approach is described by Kuntz et al., J. Mol. Biol., vol. 161, p. 269, 1982, which is incorporated by this reference herein in its entirety. One or more extant databases of crystallographic data (e.g., the Cambridge Structural Database System maintained by University Chemical Laboratory, Cambridge University, Lensfield Road, Cambridge CB2 IEW, U.K. or the Protein Data Bank maintained by Rutgers University) can then be searched for chemical compounds that approximate the shape thus defined. Chemical compounds identified by the geometric approach can be modified to satisfy criteria associated with chemical complementarity, such as hydrogen bonding, ionic interactions or Van der Waals interactions.

In some embodiments, a therapeutic composition of the present invention can comprise one or more therapeutic compounds. A therapeutic composition of the present invention can be used to treat disease in a subject such as, for example, a human in need of treatment, by administering such composition to the subject. Non-limiting examples of subjects for treatment include mammals including humans and companion animals such as cats and dogs, marsupials, reptiles and birds, food animals, zoo animals and other economically relevant animals (e.g., race horses and animals valued for their coats, such as chinchillas and minks). Additional examples of subject animals for treatment include horses, cattle, sheep, swine, chickens, turkeys. A therapeutic composition of the present invention can also include an excipient, an adjuvant and/or carrier. Suitable excipients include compounds that the animal to be treated can tolerate. Examples of such excipients include water, saline, Ringer's solution, dextrose solution, Hank's solution, and other aqueous physiologically balanced salt solutions. Nonaqueous vehicles, such as fixed oils, sesame oil, ethyl oleate, or triglycerides can also be used. Other formulations include suspensions containing viscosity enhancing agents, such as sodium carboxymethylcellulose, sorbitol, or dextran. Excipients can also contain minor amounts of additives, such as substances that enhance isotonicity and chemical stability. Examples of buffers include phosphate buffer, bicarbonate buffer and Tris buffer, while examples of preservatives include thimerosal, o-cresol, formalin and benzyl alcohol. Standard formulations can either be liquid injectables or solids which can be taken up in a suitable liquid as a suspension or solution for injection. Thus, in a non-liquid formulation, the excipient can comprise dextrose, human serum albumin, preservatives, etc., to which sterile water or saline can be added prior to administration.

In one embodiment of the present invention, a therapeutic composition can include a carrier. Carriers include compounds that increase the half-life of a therapeutic composition in the treated subject. Suitable carriers include, but are not limited to, polymeric controlled release vehicles, biodegradable implants, liposomes, bacteria, viruses, other cells, oils, esters, and glycols.

Acceptable protocols to administer therapeutic compositions of the present invention in an effective manner include individual dose size, number of doses, frequency of dose administration, and mode of administration. Determination of such protocols can be accomplished by those skilled in the art. Modes of administration can include, but are not limited to, subcutaneous, intradermal, intravenous, intranasal, oral, transdermal, intraocular and intramuscular routes.

### EXAMPLES

The following specific examples are illustrative and are not intended to limit the scope of the claims. The description of a composition, article or method in an example does not imply that the composition or article has, or has not, been produced or that a described method has, or has not, been performed, irrespective of verb tense used.

### Example 1

This example illustrates structure determination.

The crystal structure of human NMPRTase was determined at 2.7 Å resolution by the selenomethionyl single-wavelength anomalous diffraction (SAD) method (Hendrickson, W. A. (1991). Determination of macromolecular structures from anomalous diffraction of synchrotron radiation. Science 254, 51-58). Human NMPRTase contains only 2 methionine residues out of a total of 490 residues (excluding the initiator Met residue). We also obtained crystals of murine NMPRTase, but it contains only 1 methionine residue. Expectedly, the Se anomalous signal was very small based on data collected for such selenomethionyl crystals. To increase the Se anomalous signal, we introduced Met residues at several positions in human NMPRTase by site-specific mutagenesis, and succeeded in crystallizing the F132M/I151M double mutant. Surprisingly, the Se anomalous signal for this mutant crystal was still very small, only about 0.2%. Nonetheless, we were able to locate the 8 Se positions for the two NMPRTase molecules in the crystallographic asymmetric unit. After two-fold non-crystallographic symmetry (NCS) averaging, the electron density map could be readily interpreted based on the amino acid sequence of NMPRTase.

### Example 2

This example illustrates determination of binding modes.

To determine the binding modes of the reaction product NMN and the potent inhibitor FK866, wild-type human NMPRTase was co-crystallized with these compounds. The structures of the complexes were determined by the molecular replacement method. Clear electron density was observed for the compounds in all the NMPRTase molecules in the crystallographic asymmetric unit. The structure of the free enzyme of murine NMPRTase was determined by the molecular replacement method using the structure of human NMPRTase as the search model.

### Example 3

This example illustrates overall structure of human NMPRTase monomer.

The crystal structure of human NMPRTase in complex with the product NMN has been determined at 2.2 Å resolution. The current atomic model contains residues 9-41 and 54-484 for the six NMPRTase molecules in the asymmetric unit. The expression construct contains the full-length NMPRTase protein, suggesting that residues 42-53 and those at the extreme N- and C-termini (and the C-terminal histidine tag) are disordered. The atomic model has good agreement with the observed diffraction data (R factor of 20.1%) and the expected bond lengths (rms deviation of 0.006 Å) and bond angles (rms deviation of 1.4°). The majority of the residues (90%) are located in the most favored region of the Ramachandran plot. The crystallographic information is provided in Table 5.

The crystal structure of human NMPRTase in complex with the FK866 inhibitor has been determined at 2.1 Å resolution, and the crystal structure of the free enzyme of murine NMPRTase has been determined at 2.1 Å resolution (Table 3). The amino acid sequences of human and murine NMPRTase share 96% identity. Therefore the structure of the free enzyme of murine NMPRTase should also be a good model for that of human NMPRTase.

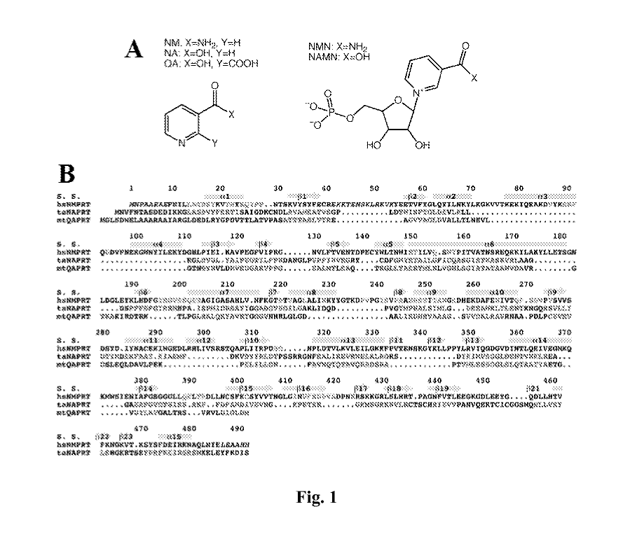

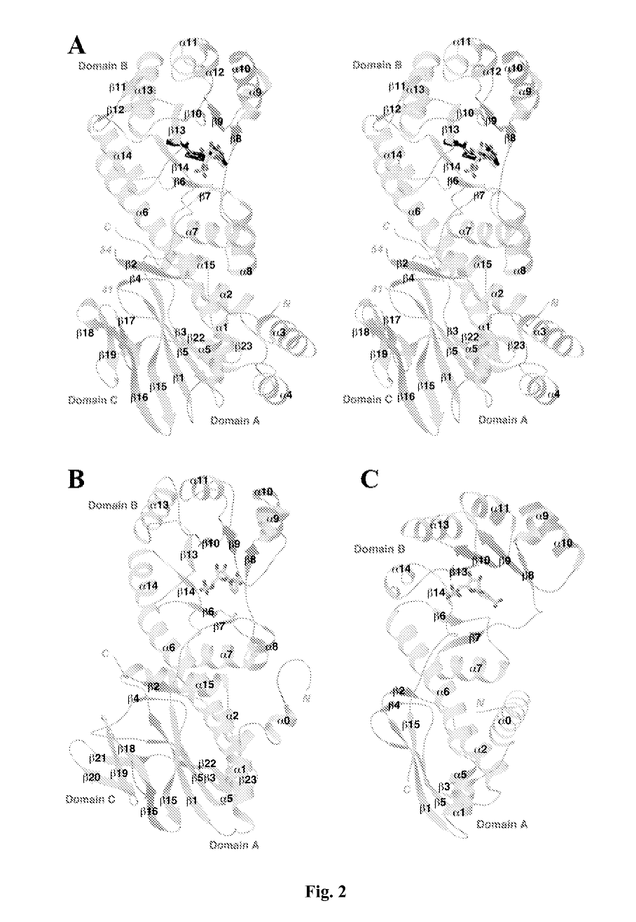

The structure of the NMPRTase monomer contains 22 b-strands (b1-b19, b21-b23) and 15 a-helices (a1-a15) (FIG. 1B), and can be divided into three domains, A, B and C (FIG. 2A). Domain A consists of a seven-stranded fully anti-parallel b-sheet with five helices on one face. Residues from both the N- and C-terminal regions of NMPRTase (9-148, 391-427, 459-494) belong to this domain, with the N-terminal region contributing three of the seven strands in the b-sheet. Domain B (residues 181-390) contains a seven-stranded a/b core. Helix a6, with 9 turns (residues 149-180), connects the two domains (FIG. 2A). Domain C contains a three-stranded anti-parallel b-sheet, and covers up the open face of the b-sheet in Domain A.

The crystallographic asymmetric units of these crystals contain two or six molecules of NMPRTase. The monomers in each of these crystals have essentially the same conformation, with rms distance of about 0.4 Å for equivalent Ca atoms. This indicates that there are no major conformational differences among the NMPRTase molecules in the same crystal.

### Example 4

This example illustrates structural comparisons between NMPRTase, NAPRTase and QAPRTase.

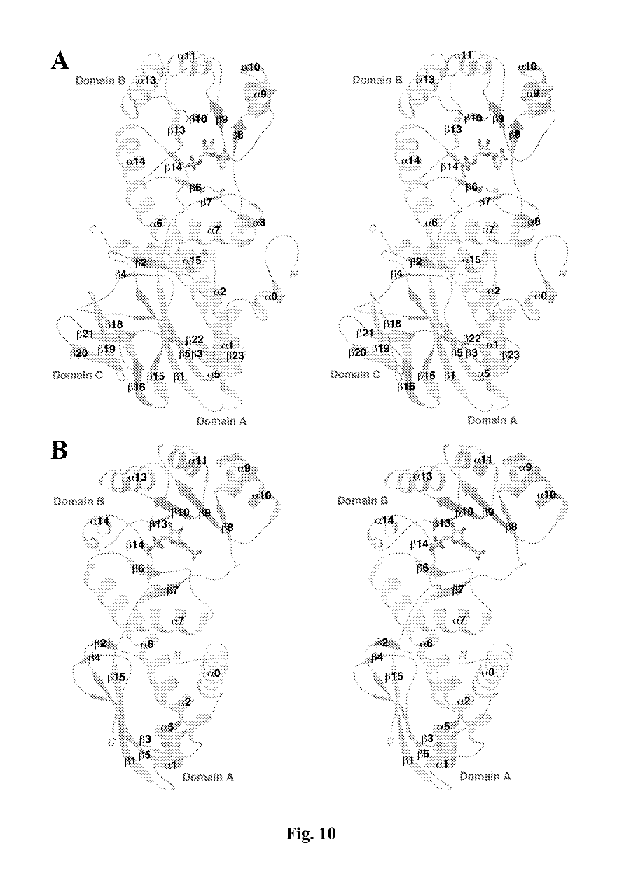

Despite sharing very limited sequence homology with QAPRTase and NAPRTase, the overall structure of human NMPRTase shows remarkable similarity to these other enzymes. The closest structural homolog is the NAPRTase from Thermoplasma acidophilum, taNAPRTase (Shin et al., 2005) (FIG. 2B, and FIG. 10), and many of the secondary structure elements in NMPRTase have structural equivalents in taNAPRTase (FIG. 1B). NMPRTase also shows structural similarity to the QAPRTases, for example that from Mycobacterium tuberculosis, mtQAPRTase (Sharma et al., 1998) (FIG. 2C, and FIG. 10). The sequence identity of the structurally-equivalent residues is about 19% between NMPRTase and these other two enzymes. However, the overall sequence identity between these enzymes is much lower (less than 10%). To facilitate the structural comparisons, the secondary structure elements in these other enzymes are named using the same scheme as in NMPRTase (FIG. 1B).

While NMPRTase shares overall structural similarity to NAPRTase and QAPRTase, there are also significant conformational differences between NMPRTase and these other two PRTases. NMPRTase contains about 100 more amino acid residues as compared to taNAPRTase. These additional residues are distributed over many regions of the structure (FIG. 1B), especially the insertion of helices a3 and a4 in domain A and strands b11 and b12 in domain B (FIG. 2A). The mtQAPRTase is even smaller in size, such that the b-sheet in its domain A contains only four strands, and domain C is absent (FIG. 2C). QAPRTase is unique in containing a long helix (a0) at its N-terminus.

In taNAPRTase, domain C consists of a four-stranded zinc knuckle-like structure (FIG. 2B), with four Cys residues being the putative ligands of a zinc ion (Shin et al., 2005). In comparison, domain C in NMPRTase contains only three b-strands (FIG. 2A). It does not have the zinc-knuckle fold, and it lacks any Cys residues.

There are also differences in the orientation of domain B relative to domain A in these structures. With domain A of NMPRTase and taNAPRTase placed in the same orientation, the orientation of domain B differs by about 13° between the two structures (FIG. 2B). The difference is even larger, about 34°, for the orientation of domain B in mtQAPRTase (FIG. 2C). These organizational differences between the two domains have significant impact on the dimerization and the composition of the active site of these enzymes (see next section).

The crystal structure of yeast NAPRTase (scNAPRTase) was reported recently (Chappie et al., 2005). The domain organization in this structure is entirely different from that of NMPRTase and the bacterial NAPRTase and QAPRTases. Domain A caps the open end of the b-sheet of domain B in scNAPRTase, which exists as monomers in solution. This organization may be unique to scNAPRTase, however, as our structure of yeast QAPRTase, also known as BNA6, shows a domain organization that is similar to mtQAPRTase.

### Example 5

This example illustrates that human NMPRTase is a dimer.

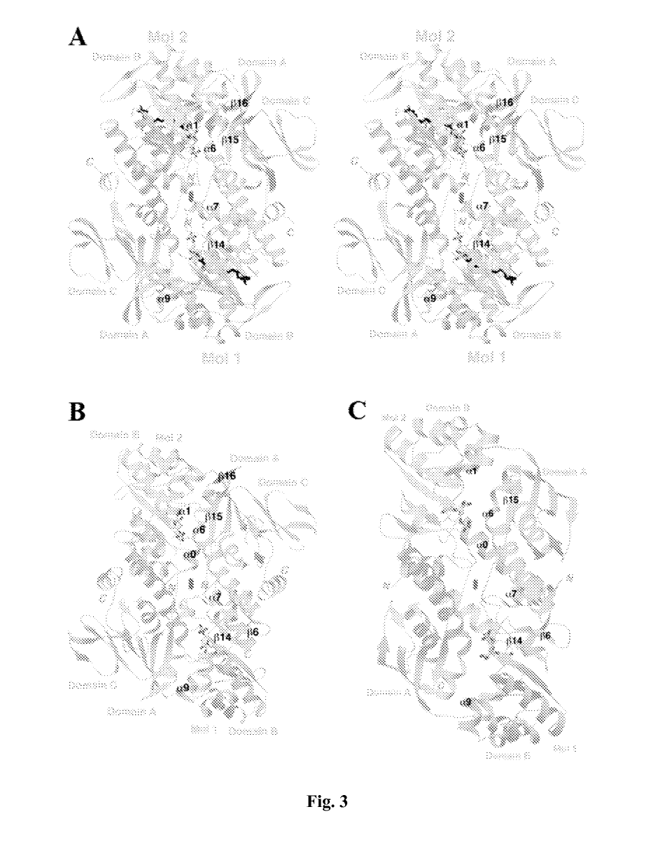

Our gel-filtration and light-scattering studies show that human and murine NMPRTase is dimeric in solution. The crystal structures reveal an intimately associated dimer of both human and murine NMPRTase monomers (FIG. 3A), with 4000 Å² of the surface area of each monomer being buried at the dimer interface. The two monomers are arranged in a head-to-tail fashion, with domain A in one monomer contacting domain B in the other monomer. Domain C is located far from the dimer interface, and does not appear to help stabilize the dimer.

Residues in domain A that make large contributions to the surface area burial in the dimer interface are located in several helices (a1, a3, a5, and a6) and two strands (b15 and b16) (FIG. 1B and FIG. 3A). These residues interact with those near the top of the b-sheet in domain B of the other monomer (FIG. 3A). In addition, residues in the loop connecting domains A and B (b14-b15 loop) are located near the two-fold axis of the dimer (FIG. 3A).

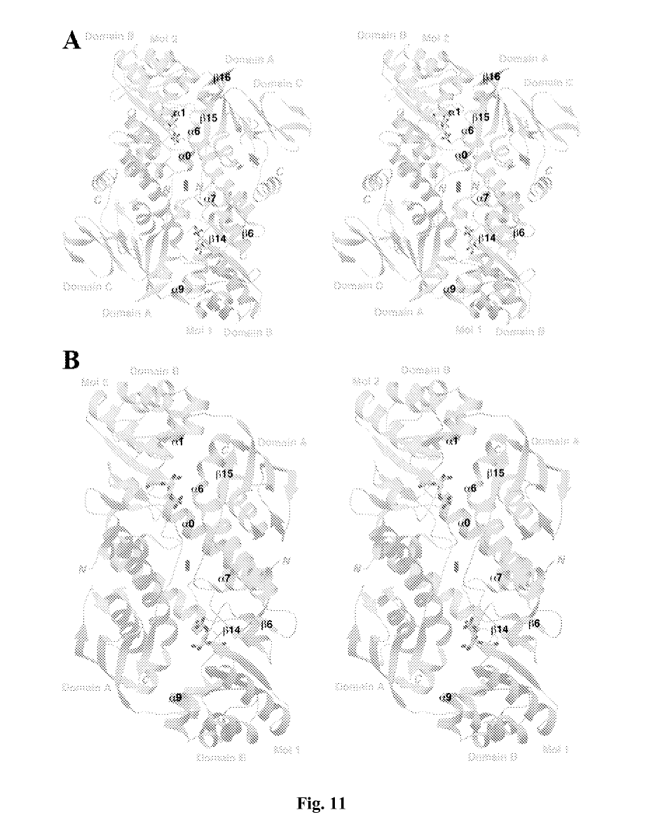

taNAPRTase and mtQAPRTase also use a head-to-tail arrangement to form their dimers (FIGS. 3B and 3C, and FIG. 11). However, because of the differences in the relative positions of domains A and B in these enzymes, there are significant differences in their dimer organization compared to NMPRTase. This is especially true for mtQAPRTase, where helix a1 is no longer involved in dimer formation (FIG. 3C). This has dramatic impact on the composition of the active site of the enzymes, as a Tyr residue in helix a1 helps recognize the NM and NA substrate in NMPRTase and NAPRTase (see next). Moreover, taNAPRTase and mtQAPRTase can exist as hexameric rings, whereas NMPRTase is only a dimer. The larger size of NMPRTase precludes the formation of the hexameric structure due to steric clashes among the dimers.

### Example 6

This example illustrates the binding mode of NMN to NMPRTase.

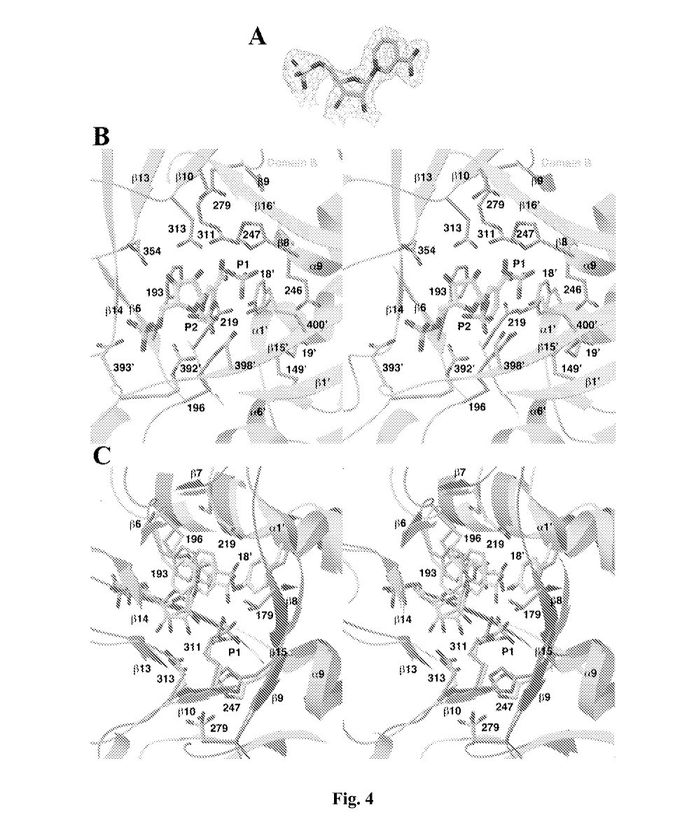

The active site of NMPRTase is revealed by the structure of the complex with the reaction product NMN (FIG. 4A). NMN is bound near the top of the central b-sheet in domain B (FIG. 2A). Moreover, the active site is located in the dimer interface, with several residues from the other monomer of the dimer having critical roles in recognizing the NMN molecule (FIG. 4B). Ten different segments of NMPRTase are involved in forming the active site. Seven of these come from residues near the ends of the seven b-strands in the central b-sheet of domain B in one monomer, whereas the remaining three segments come from the second monomer (helices a1 and a6, and strand b15) (FIG. 4B). Therefore NMPRTase can only be active in its dimeric form.

The nicotinamide ring of NMN is sandwiched between the side chain of Phe¹⁹³ at the end of strand b6 of one monomer and that of Tyr18′ in helix al of the other monomer (with the prime indicating the second monomer), showing p-stacking interactions (FIG. 4B). The carbonyl oxygen of the amide group of NMN is pointed towards the side chain of Arg³¹¹ (in strand b10), although direct hydrogen-bonding interactions are unlikely as the plane of the guanidinium group is perpendicular to the carbonyl group. The amide nitrogen is hydrogen-bonded to the side chain of Asp²¹⁹ (b7).

The ribose and the phosphate groups of NMN are located in a highly hydrophilic binding pocket, with more than ten charged residues (FIG. 4B). The two hydroxyl groups on the ribose are hydrogen-bonded to the side chains of Arg³¹¹, Asp³¹³ (b10), and Asp³⁵⁴ (b13). One of the terminal oxygen atoms of the phosphate group of NMN is hydrogen-bonded to the main-chain amide of Gly³⁸⁴ (b14-b15 loop), and another oxygen is hydrogen-bonded to the side chain of Arg^{392′} (b14-b15 loop) from the other monomer (FIG. 4B).

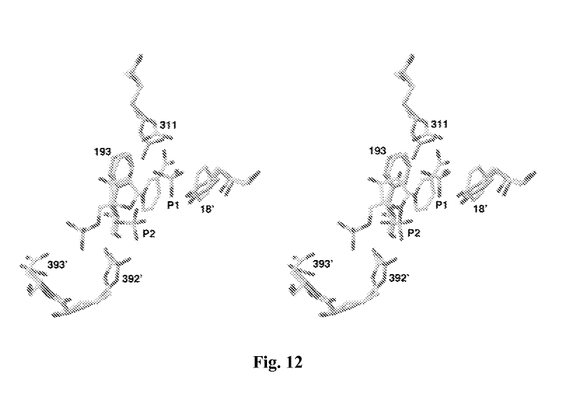

Structural comparisons between the NMN complex of human NMPRTase and the free enzyme of murine NMPRTase show that there is no overall conformational change in the enzyme upon NMN binding. The rms distance between 924 equivalent Ca atoms of the two dimers is 0.4 Å. Most of the residues in the active site have similar conformations in the free enzyme and the NMN complex. However, the side chain of Tyr18′ assumes a different rotamer in the free enzyme (a change of 70° in the c2 torsion angle), which would collide with the nicotinamide ring of NMN (FIG. 12). In addition, the position of the guanidinium group of Arg³¹¹ in the free enzyme would clash with the ribose of NMN, and this side chain assumes a different conformation in the complex (FIG. 12). This suggests that adjustments in the side chain conformation of these residues may be needed for NMN binding.

Our structural analyses revealed the presence of two free phosphate groups near the NMN molecule (the crystallization solution contained 50 mM phosphate). The first one (P1) is directly hydrogen-bonded to the side chains of Arg¹⁹⁶, His²⁴⁷, Arg³¹¹, and Tyr^{18′} (FIG. 4B). This phosphate group is also hydrogen-bonded to the 2′-hydroxyl of the ribose in NMN. The second phosphate (P2) is not as ordered, and interacts with the side chains of Arg¹⁹⁶, Arg^{392′} and Lys^{400′} (FIG. 4B).

Remarkably, His²⁴⁷ is equivalent to a His residue that is auto-phosphorylated in the NAPRTases (Gross, J., Rajavel, M., Segura, E., and Grubmeyer, C. (1996) Energy coupling in Salmonella typhimurium nicotinic acid phosphoribosyltransferase: identification of His-219 as site of phosphorylation. Biochem 35, 3917-3924). In the presence of ATP, NAPRTases catalyze the formation of NAMN with concomitant hydrolysis of ATP, through the formation of a phosphohistidine intermediate (Gross, J. W., Rajavel, M., and Grubmeyer, C. (1998). Kinetic mechanism of nicotinic acid phosphoribosyltransferase: implications for energy coupling. Biochem 37, 4189-4199). Phosphorylated NAPRTase has higher catalytic activity and lower Kₘ for the substrates. Our structure suggests that the P1 phosphate could be a mimic for a phosphorylated His residue, raising the tantalizing possibility that mammalian NMPRTase could also be activated by auto-phosphorylation.

### Example 7

This example illustrates the molecular basis for substrate specificity.

Our structure of NMPRTase is the first one for this class of enzymes, and comparison to that of NAPRTase allowed us to examine the molecular basis for the substrate specificity of these enzymes. The overall binding mode of NMN to NMPRTase is similar to that of NAMN to taNAPRTase (Shin et al., 2005) (FIG. 4C). However, there are three major structural differences near the amide group of NMN between NMPRTase and taNAPRTase. First of all, the side chain of the negatively charged Asp219 residue is directly hydrogen-bonded to the amide group of NMN in NMPRTase (FIG. 4C). The presence of this negative charge should disfavor the binding of NA to the active site of NMPRTase. In fact, NAPRTases have a Ser residue at the equivalent position (FIG. 1B), and the side chain of this Ser residue in not involved in NA binding (FIG. 4C). Secondly, the negative charge on the carboxylate group of NA is recognized by the side chain of Arg²³⁵ in taNAPRTase (FIG. 4C). This residue is equivalent to Arg311 in NMPRTase, which does not have optimal interactions with the amide group of NM. Instead, the positive charge in its side chain is involved in the binding of a phosphate group (FIG. 4B).

Finally, NMPRTase contains a 10-residue insertion in the loop connecting helix a8 and strand b8 (FIG. 1B). As a result, there are significant conformational differences between NMPRTase and taNAPRTase in this region. One of the carboxylate oxygens of NA is hydrogen-bonded to the side chain of Thr¹⁷⁹ in strand b8 of taNAPRTase (FIG. 4C). In NMPRTase, strand b8 has moved away by about 2 Å, such that there are no direct interactions between NM and residues in this strand. This structural difference may also have crucial implications for inhibitor selectivity as well.

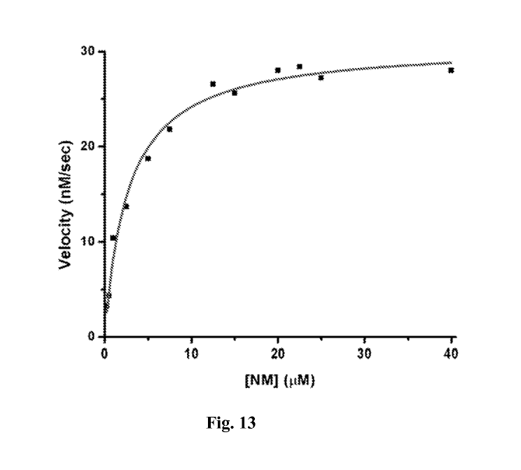

In kinetic experiments, our purified wild-type NMPRTase demonstrated robust activity towards the NM substrate. The Kₘ value based in our assays is about 2 mM (FIG. 13), which is the same as that reported earlier (Revollo et al., 2004; Rongvaux et al., 2002).

### Example 8

This example illustrates the binding mode of FK866.

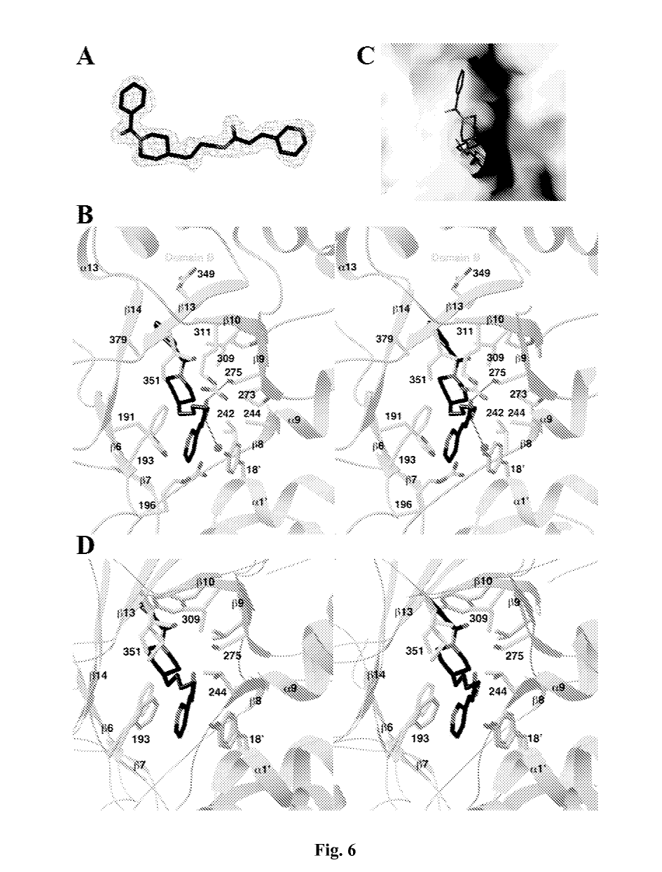

FK866 has clearly defined electron density from the crystallographic analysis (FIG. 6A). The compound is located in the center of the parallel b-sheet in domain B, having mostly van der Waals interactions with NMPRTase (FIG. 6B). The binding site is located in the dimer interface of the enzyme. The second monomer helps close off the open side of the b-sheet in domain B, producing a tunnel that extends through the dimer. The FK866 compound is situated in this tunnel (FIG. 6C). In the free enzyme, the tunnel is occupied by several water molecules.

The pyridyl ring of the inhibitor is sandwiched between the side chains of Phe¹⁹³ of one monomer and Tyr^{18′} of the other monomer, showing p-stacking interactions (FIG. 6B). The carbonyl oxygen atom of the amide bond near the center of the inhibitor is hydrogen-bonded to the side chain hydroxyl of Ser²⁷⁵ (in strand b9), while the amide nitrogen is hydrogen-bonded to a water molecule. This water is also hydrogen-bonded to the side chains of Asp²¹⁹ and Ser²⁴¹, as well as the main-chain carbonyl oxygen of residue 242. The aliphatic carbon atoms of FK866 interact with the mostly hydrophobic side chains in the center of the b-sheet of domain B (FIG. 6B). At the other end of the inhibitor, the phenyl ring is situated in a shallow groove on the surface of NMPRTase (FIG. 6C). However, this and the piperidine rings, as well as the amide bond linking them, are mostly exposed to solvent.

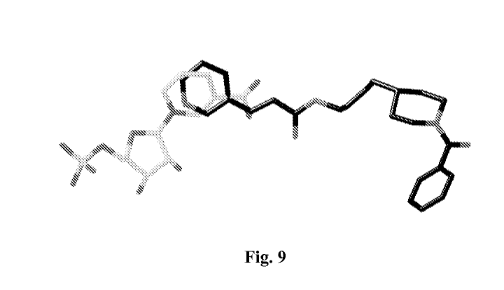

There are no overall conformational changes in the enzyme upon inhibitor binding. The side chain of Tyr^{18′} assumes a different rotamer in the free enzyme (as discussed above for NMN), which would collide with the pyridyl ring of FK866 (FIG. 9). At the other end of the inhibitor, the side chain of Tyr²⁴⁰ assumes a different rotamer in the free enzyme (c1 change of 150°), which could clash with the piperidine ring (FIG. 9).

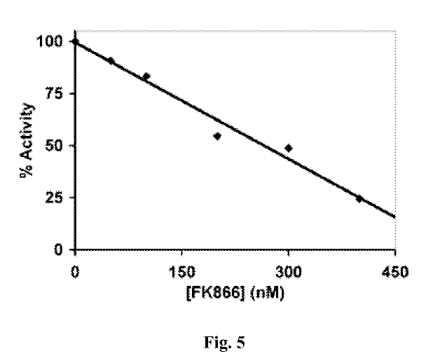

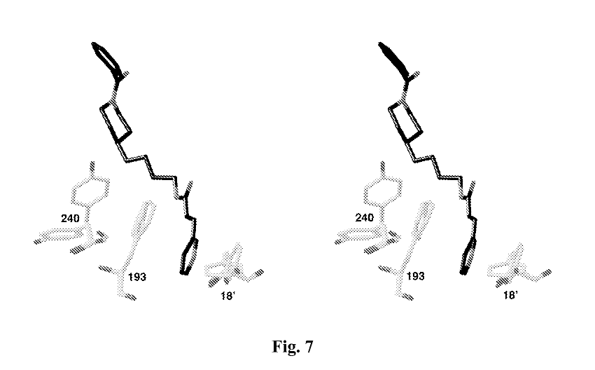

The bound position of the pyridyl ring of FK866 is essentially the same as that of the nicotinamide ring of NMN (FIG. 2A, FIG. 7). Therefore, our structure would predict that the compound FK866 should be competitive versus the NM substrate of the enzyme. Interestingly, a noncompetitive inhibition mechanism was suggested based on earlier kinetic studies (Hasmann and Schemainda, 2003). Our structural information however suggests a different interpretation for the kinetic data. The high potency of FK866 towards NMPRTase (Kᵢ of 0.4 nM) suggests that it is likely to have a very slow rate of dissociation from the enzyme, such that FK866 essentially functions as an irreversible inhibitor during the kinetic assays. Therefore, the reduction in Vₘₐₓ in the presence of the inhibitor is due simply to the removal of active enzyme into the inactive and non-dissociable enzyme:FK866 complex. This would predict a linear relationship between FK866 concentration and the Vₘₐₓ, Vₘₐₓ=k_{cat}([E]−[I]), if all the inhibitor is bound to the enzyme. Our kinetic data is entirely in agreement with this model (FIG. 5). moreover, the total enzyme concentration estimated from this model (vertical intercept/-slope) is 540 nM, essentially the same as the amount of enzyme that we put in the assay. A re-plotting of the kinetic data reported earlier also gives a linear relationship between Vₘₐₓ and FK866 concentration (data not shown).

### Example 9

This example illustrates molecular basis for the specificity of FK866 towards NMPRTase.

The structural information suggests the molecular basis for the specificity of FK866 for NMPRTase. The largest structural difference between NMPRTase and taNAPRTase in the binding site for FK866 is for residues in strand b8 (FIG. 6D), due to the insertion of 10 residues in NMPRTase (FIG. 1B) as discussed for NMN binding above. This strand is placed closer to the center of the b-sheet in domain B in taNAPRTase. Moreover, the side chain of Thr¹⁷⁹ in b8 is pointed towards the tunnel in taNAPRTase, whereas the equivalent residue in NMPRTase is Ala²⁴⁴ (FIG. 1B). This Thr side chain of taNAPRTase actually clashes with FK866 in the NMPRTase complex (FIG. 6D). In addition, residue Ile³⁵¹ in NMPRTase is replaced by Met in taNAPRTase, further reducing the size of the tunnel. The overall result of these structural differences is that taNAPRTase does not contain a tunnel at the center of the b-sheet in domain B, which is the molecular basis why FK866 does not inhibit NAPRTases.

NMPRTase is a crucial enzyme in the salvage pathway of NAD⁺ biosynthesis, and has important functions in regulating NAD⁺ levels in cells undergoing significant NAD⁺ turnover. Tumor cells have elevated NAD⁺ turnover due to higher ADP-ribosylation activity. FK866 is a potent inhibitor of NMPRTase and can reduce NAD⁺ levels and cause apoptosis of tumor cells (Hasmann and Schemainda, 2003), validating NMPRTase as a target for the development of novel anti-cancer agents. On the other hand, elevated NAD⁺ biosynthesis may protect against neurodegeneration (Araki et al., 2004). Our studies define the three-dimensional structure of human and murine NMPRTase, reveal the molecular mechanism for the substrate specificity of this enzyme, and define the binding mode of FK866 and the structural basis for its specificity for NMPRTase. These results provide a foundation of developing and optimizing new inhibitors against this important enzyme.

### Example 10

This example illustrates protein expression and purification.

Full-length human and murine NMPRTase (residues 1-491) was sub-cloned into the pET26b vector (Novagen) and over-expressed in E. coli at 20° C. The expression construct introduced a hexa-histidine tag at the C terminus. After cell lysis, the soluble protein was purified by nickel-agarose affinity chromatography, anion exchange and gel filtration chromatography. The protein was concentrated to 30 mg/ml in a buffer containing 20 mM Tris (pH 7.9), 200 mM NaCl, 5 mM dithiothreitol (DTT), and 5% (v/v) glycerol and stored at −80° C. The C-terminal His tag was not removed for crystallization.

The seleno-methionyl protein was produced in B834(DE3) cells (Novagen), grown in defined LeMaster media supplemented with seleno-methionine (Hendrickson et al., 1990), and purified following the same protocol as that for the native protein. The seleno-methionyl protein was concentrated to 20 mg/ml in a buffer of 20 mM Tris (pH 7.9), 200 mM NaCl, 5% (v/v) glycerol, and 5 mM DTT.

To increase the Se anomalous diffraction signal, site-specific mutants of NMPRTase were created to introduce additional Met residues into the protein. Based on sequence alignment, the following mutations were designed: L⁶²M, I⁶⁵M, F¹³²M, I¹⁵¹M, and I²⁶⁵M. The mutants were created with the QuikChange kit (Stratagene), and sequenced to confirm the incorporation of the correct mutation. We screened seven different combinations of the mutation sites, as double, triple and quintuple mutants, and found that the F¹³²M/I¹⁵¹M double mutant could be crystallized. Atomic coordinates obtained from x-ray crystallographic analysis of such crystals are set forth in Table 4.

### Example 11

This example illustrates protein crystallization.

Crystals of the seleno-methionyl free enzyme of NMPRTase (F¹³²M/I¹⁵¹M double mutant) were grown with the sitting-drop vapor diffusion method at 22° C. The reservoir solution contained 50 mM phosphate buffer (pH 9.2), 24% (w/v) PEG3350, 200 mM NaCl, and 5 mM DTT. BaCl₂ was used as an additive in the drop solution. The crystals were cryo-protected by transferring to the reservoir solution supplemented with 15% (v/v) ethylene glycol and flash-frozen in liquid propane for data collection at 100 K. They belong to space group P2₁2₁2₁, with cell parameters of a=87.98 Å, b=93.43 Å, and c=244.26 Å. There are four molecules of NMPRTase in the asymmetric unit.

Crystals of human NMPRTase in complex with NMN were obtained at 22° C. by the sitting-drop vapor diffusion method. The protein (20 mg/ml concentration) was incubated with 2 mM NMN (protein:NMN molar ratio of 1:5) at 4° C. for 30 mins prior to crystallization setup. BaCl₂ was used as an additive in the drop solution. The reservoir solution contains 50 mM phosphate buffer (pH 9.2), 26% (w/v) PEG3350, 200 mM NaCl, and 5 mM DTT. The crystals belong to space group C2, with cell parameters of a=253.07 Å, b=101.37 Å, c=148.20 Å, and β=125.48°. There are six copies of the NMPRTase:NMN complex in the asymmetric unit (two dimers and two monomers sitting at the crystallographic two-fold axis).

Crystals of human NMPRTase in complex with FK866 were obtained at 22° C. by the sitting-drop vapor diffusion method. The protein (20 mg/ml concentration) was incubated with 2 mM FK866 (protein:inhibitor molar ratio of 1:5) at 4° C. for 30 mins prior to crystallization setup. BaCl₂ was used as an additive in the drop solution. The reservoir solution contains 50 mM phosphate buffer (pH 9.2), 26% (w/v) PEG3350, 200 mM NaCl, and 5 mM DTT. The crystals belong to space group P2₁, with cell parameters of a=60.78 Å, b=105.89 Å, c=83.43 Å, and β=96.45°. There are two copies of the NMPRTase:FK866 complex in the asymmetric unit.

Crystals of murine NMPRTase free enzyme were obtained by the sitting-drop vapor diffusion method at 4° C. The reservoir solution contained 50 mM phosphate buffer (pH 9.2), 21% (w/v) PEG3350, 200 mM NaCl, and 5 mM DTT. BaCl₂ was used as an additive in the drop solution. The crystals are essentially isomorphous to those of the human NMPRTase:FK866 complex, with cell parameters of a=60.26 Å, b=107.73 Å, c=83.28 Å, and β=96.56°.

### Example 12

This example illustrates data collection and processing.

X-ray diffraction data were collected on an ADSC CCD at the X4A beamline or an Mar imaging plate detector at the X4C beamline of Brookhaven National Laboratory. A seleno-methionyl single-wavelength anomalous diffraction (SAD) data set to 2.7 Å resolution was collected at 100K on the free enzyme crystal (F132M/I151M double mutant, in the orthorhombic system), and native reflection data sets were collected for the other crystals. The diffraction images were processed and scaled with the HKL package (Otwinowski, Z., and Minor, W. (1997) Processing of X-ray diffraction data collected in oscillation mode. Method Enzymol 276, 307-326). The data processing statistics are summarized in Table 4.

### Example 12

This example illustrates structure determination and refinement.

Despite the introduction of two additional Met residues, the Se anomalous signal was still rather small, only about 0.2%. The locations of 16 Se atoms were determined with the program BnP (Weeks, C. M., and Miller, R. (1999) The design and implementation of SnB v2.0. J Appl Cryst 32, 120-124). Reflection phases to 2.7 Å resolution were calculated based on the SAD data with the program SOLVE/RESOLVE (Terwilliger, T. C. (2003). SOLVE and RESOLVE: Automated structure solution and density modification. Meth Enzymol 374, 22-37), which built partial models for the four molecules of NMPRTase in the asymmetric unit.

The non-crystallographic symmetry (NCS) parameters were determined based on the partial models and the Se sites, and the reflection phases were improved by four-fold NCS averaging with the program DM (CCP4 (1994). The CCP4 suite: programs for protein crystallography. Acta Cryst D50, 760-763). The atomic model for NMPRTase was built with the program O (Jones, T. A., Zou, J. Y., Cowan, S. W., and Kjeldgaard, M. (1991) Improved methods for building protein models in electron density maps and the location of errors in these models. Acta Cryst A47, 110-119). After one cycle of refinement at 2.7 Å resolution with the program CNS (Brunger, A. T., Adams, P. D., Clore, G. M., DeLano, W. L., Gros, P., Grosse-Kunstleve, R. W., Jiang, J.-S., Kuszewski, J., Nilges, M., Pannu, N. S., et al. (1998). Crystallography & NMR System: A new software suite for macromolecular structure determination. Acta Cryst D54, 905-921), the model for the dimer of NMPRTase was used to solve the structure of the human NMPRTase:FK866 complex and the murine NMPRTase free enzyme by the molecular replacement method, with the program COMO (Jogl, G., Tao, X., Xu, Y., and Tong, L. (2001) COMO: A program for combined molecular replacement. Acta Cryst D57, 1127-1134). The refinement of these structures was carried out with CNS and Refmac (Murshudov, G. N., Vagin, A. A., and Dodson, E. J. (1997). Refinement of macromolecular structures by the maximum-likelihood method. Acta Cryst D53, 240-255). The refinement statistics are summarized in Table 4.

### Example 13

This example illustrates an NMPRTase assay

The catalytic activity of NMPRTase was determined using a coupled-enzyme spectrometric assay, following a published protocol (Revollo et al., 2004). Briefly, the NMN product of NMPRTase is converted to NAD⁺ with the enzyme NMN/NAMN adenylyltransferase (NMNAT), and NAD⁺ is then reduced to NADH by alcohol dehydrogenase (Sigma) using ethanol as the substrate. By monitoring the appearance of NADH at 340 nm, the activity of NMPRTase can be determined. Human NMNAT was over-expressed in E. coli and purified following a published protocol (Zhou, T., Kurnasov, O., Tomchick, D. R., Binns, D. D., Grishin, N. V., Marquez, V. E., Osterman, A. L., and Zhang, H. (2002). Structure of human nicotinamide/nicotinic acid mononucleotide adenylyltransferase. Basis for the dual substrate specificity and activation of the oncolytic agent tiazofurin. J Biol Chem 277, 13148-13154). The reaction buffer contains 50 mM Tris (pH 7.5), 0.4 mM PRPP, various concentrations of NM (or NA), 2.5 mM ATP, 12 mM MgCl₂, 1.5% ethanol, 10 mM semicarbazide (to remove the acetaldehyde product of ethanol oxidation), 0.02% BSA, 10 mg/ml NMNAT, 30 mg/ml ADH, and 0.5 mM NMPRTase. The reactions are carried out at room temperature.

### Other Embodiments

The detailed description set-forth above is provided to aid those skilled in the art in practicing the present teachings. However, the teachings described and claimed herein are not to be limited in scope by the specific embodiments herein disclosed. Any equivalent embodiments are intended to be within the scope of this invention. Various modifications of the teachings which do not depart from the spirit or scope of the present inventive discoveries, in addition to those shown and described herein, will become apparent to those skilled in the art from the foregoing description. Such modifications are also intended to fall within the scope of the appended claims.

TABLE 5
Summary of crystallographic information

| | Human | Human | Murine |
|---|---|---|---|
| | NMPRTase+ | NMPRTase+ | NMPRTase |
| Structure | NMN | FK866 | free enzyme |
| Space group | C2 | P21₁ | P2₁ |
| Number of unique NMPRTase molecules | 6 | 2 | 2 |
| Maximum resolution (Å) | 2.2 | 2.1 | 2.1 |
| Number of observations | 421,561 | 200,508 | 190,339 |
| R_{merge} (%)¹ | 7.3 (32.3) | 9.2 (23.8) | 8.1 (26.7) |
| I/□I | 14.6 (3.0) | 11.4 (3.6) | 13.5 (2.9) |
| Resolution range used for refinement (Å) | 30-2.2 | 83-2.1 | 83-2.1 |
| Number of reflections | 142,302 | 57,379 | 55,602 |
| Completeness (%) | 93 (76) | 98 (77) | 95 (77) |
| R factor (%)² | 20.1 (27.5) | 24.1 (25.5) | 21.9 (23.0) |
| Free R factor (%) | 24.9 (32.5) | 29.3 (35.2) | 26.3 (33.0) |
| rms deviation in bond lengths (Å) | 0.006 | 0.010 | 0.010 |
| rms deviation in bond angles (°) | 1.4 | 1.2 | 1.2 |

$1. {R}_{merge} = {\sum }_{h} {\sum }_{i} \vert {I}_{hi} - 〈 {I}_{h} 〉 \vert / {\sum }_{h} {\sum }_{i} {I}_{hi} . The numbers in parentheses$ $are for the highest resolution shell .$
$2. R = {\sum }_{h} \vert {F}_{h}^{o} - {F}_{h}^{c} \vert / {\sum }_{h} {F}_{h}^{o} .$

What is claimed is:

1. A crystal comprising a complex, wherein the complex comprises a nicotinamide phosphoribosyltransferase (NMPRTase) and at least one ligand of NMPRTase.

2. A crystal in accordance with claim 1, wherein the NPRTase is a human NMPRTase and the at least one ligand is nicotinamide mononucleotide (NMN).

3. A crystal in accordance with claim 2, wherein the crystal belongs to space group C2.

4. A crystal in accordance with claim 3, wherein the crystal has cell parameters of a=253.07 Å, b=101.37 Å, c=148.20 Å, and β=125.48°.

5. A crystal in accordance with claim 4, wherein the crystal comprises an asymmetric unit comprising six copies of the NMPRTase:NMN complex.

6. A crystal in accordance with claim 5, wherein the asymmetric unit comprises two dimers and two monomers at a crystallographic two-fold axis.

7. A crystal in accordance with claim 2, wherein the crystal further comprises a first free phosphate group hydrogen bonded to the side chains of Arg¹⁹⁶, His²⁴⁷, Arg³¹¹ and Tyr^{18,} of the NMPRTase, and a second free phosphate hydrogen bonded to to the 2′-hydroxyl of the ribose of the NMN.

8. A crystal in accordance with claim 2, wherein a nicotinamide ring of the NMN exhibits pi-stacking interaction with Phe¹⁹³ and Tyr^{18′} of the NMPRTase.

9. A crystal in accordance with claim 1, wherein the NMPRTase is a human NMPRTase and the at least one ligand is an NMPRTase inhibitor.

10. A crystal in accordance with claim 9, wherein the NMPRTase inhibitor is (E)-N-[4-(1-benzoylpiperidin-4-yl) butyl]-3-(pyridin-3-yl) acrylamide (FK866).

11. A crystal in accordance with claim 9, wherein the crystal belongs to space group P2₁.

12. A crystal in accordance with claim 11, wherein the crystal has cell parameters of a=60.78 Å, b=105.89 Å, c=83.43 Å, and β=96.45°.

13. A crystal in accordance with claim 12, wherein the crystal comprises an asymmetric unit comprising two copies of the NMPRTase:FK866 complex.

14. A crystal in accordance with claim 9, wherein the crystal is sufficiently pure to determine atomic coordinates of the NMPRTase protein by X-ray diffraction to a resolution of about 2.1 Å.

15. A crystal in accordance with claim 9, wherein the inhibitor is hydrogen-bonded to the side chain hydroxyl of Ser²⁷⁵ of the NMPRTase.

16. A crystal in accordance with claim 9 or 10, wherein the NMPRTase inhibitor comprises an aromatic ring which exhibits pi-stacking interaction with Phe¹⁹³ and Tyr^{18′} of the NMPRTase.

17. A crystal in accordance with claim 10, wherein the crystal further comprises a water molecule, wherein the water molecule is hydrogen bonded to the amide nitrogen of the FK866 and to the side chains of Asp²¹⁹ and Ser²⁴¹ of the NMPRTase.

18. A crystal in accordance with claim 1, wherein the NMPRTase is a human NMPRTase comprising the sequence set forth in SEQ ID NO: 1.

19. A crystal comprising a substantially pure murine nicotinamide phosphoribosyltransferase (NMPRTase).

20. A crystal in accordance with claim 19, wherein the crystal has cell parameters of a=60.26 Å, b=107.73 Å, c=83.28 Å, and β=96.56°.

21. A crystal in accordance with claim 19, wherein the crystal comprises an asymmetric unit substantially isomorphic to a unit of a human NMPRTase:FK866 complex.

22. A crystal in accordance with claim 19, wherein the crystal is sufficiently pure to determine atomic coordinates of the NMPRTase protein by X-ray diffraction to a resolution of about 2.1 Å.

24. A crystal in accordance with claim 19, wherein the murine NMPRTase has the sequence set forth in SEQ ID NO: 2.

25. A crystal comprising a substantially pure human nicotinamide phosphoribosyltransferase (NMPRTase).

26. A crystal in accordance with claim 25, wherein the crystal is sufficiently pure to determine atomic coordinates of the NMPRTase by X-ray diffraction to a resolution of about 2.7 Å.

27. A crystal comprising an F¹³²M/I¹⁵¹M double mutant of human nicotinamide phosphorybosyltransferase (NMPRTase) enzyme.

28. A crystal in accordance with claim 27, wherein the crystal belongs to space group P2₁2₁2₁.

29. A crystal in accordance with claim 28, wherein the crystal has cell parameters of a=87.98 Å, b=93.43 Å, and c=244.26 Å.

30. A crystal in accordance with claim 29, wherein the crystal comprises an asymmetric unit comprising four molecules of the NMPRTase.

31. A method of forming a nicotinamide phosphoribosyltransferase (NMPRTase) crystal, the method comprising:
(a) expressing an NMPRTase in cells;
(b) purifying the NMPRTase expressed in (a); and
(c) subjecting the NMPRTase purified in (b) to crystallizing conditions.

32. A method in accordance with claim 31, wherein the NMPRTase is a human or murine NMPRTase.

33. A method in accordance with claim 31, wherein the NMPRTase is an F¹³²M/I¹⁵¹M double mutant of a human NMPRTase.

34. A method in accordance with claim 31, wherein the cells are E. coli cells.

35. A method in accordance with claim 31, wherein the NMPRTase comprises a carboxy-terminal histidine tag.

36. A method in accordance with claim 35, wherein the purifying the NMPRTase comprises subjecting a lysate from the cells to nickel-agarose chromatography, anion exchange chromatography and gel filtration chromatography.

37. A method in accordance with claim 31, further comprising incubating the NMPRTase purified in (b) with at least one NMPRTase ligand prior to (c).

38. A method in accordance with claim 37, wherein the at least one NMPRTase ligand is nicotinamide mononucleotide (NMN).

39. A method in accordance with claim 37, wherein the at least one NMPRTase ligand is (E)-N-[4-(1-benzoylpiperidin-4-yl)butyl]-3-(pyridin-3-yl)acrylamide (FK866).

40. A method in accordance with claim 31, wherein subjecting the NMPRTase to crystallizing conditions comprises subjecting the NMPRTase to sitting-drop vapor diffusion.

41. A method of identifying a compound that modifies nicotinamide phosphoribosyltransferase (NMPRTase) activity, the method comprising:
(a) designing a candidate compound predicted to form at least one bond with an NMPRTase, wherein the designing comprises computer-aided design using atomic coordinates of an NMPRTase or an NMPRTase-ligand complex;
(b) obtaining the candidate compound;
(c) contacting an NMPRTase with the candidate compound in vitro; and
(d) detecting inhibition or enhancement of NMPRTase activity.

42. The method of claim 41, wherein the atomic coordinates of an NMPRTase or an NMPRTase-ligand complex are set forth in at least one table selected from the group consisting of Table 1, Table 2 and Table 3.

43. A method in accordance with claim 41, wherein the candidate compound is a candidate NMPRTase inhibitor.

44. A method in accordance with claim 43, wherein the inhibitor binding site of an NMPRTase is a binding site for (E)-N-[4-(1-benzoylpiperidin-4-yl)butyl]-3-(pyridin-3-yl)acrylamide (FK866).

45. A method in accordance with claim 41, wherein the candidate compound is a candidate NMPRTase activity enhancer.

46. A method in accordance with claim 41, wherein obtaining the candidate compound comprises synthesizing the candidate compound.

47. A method in accordance with claim 41, wherein the candidate compound predicted to form at least one bond with an NMPRTase is a candidate compound predicted to form at least one bond with an amino acid selected from the group consisting of Arg311, Asp313, Asp354, Gly384 and Arg392′.

48. A method in accordance with claim 41, wherein the candidate compound predicted to form at least one bond with an NMPRTase is a candidate compound predicted to form at least one bond with at least one amino acid defining a substrate-binding pocket, wherein the at least one amino acid is selected from the group consisting of, in a first monomer of a human NMPRTase dimer, His¹⁹¹, Asp¹⁹², Phe¹⁹³, Gly¹⁹⁴, Tyr¹⁹⁵, Arg¹⁹⁶, Gly¹⁹⁷, Val¹⁹⁸, Ser¹⁹⁹, Gly²¹⁷, Thr²¹⁸, Asp²¹⁹, Thr²²⁰, Val²²¹, Tyr²⁴⁰, Ser²⁴¹, Val²⁴², Pro²⁴³, Ala²⁴⁴, Ala²⁴⁵, Glu²⁴⁶, His²⁴⁷, Ser²⁴⁸, Val²⁷⁴, Ser²⁷⁵, Val²⁷⁶, Val²⁷⁷, Ser²⁷⁸, Asp²⁷⁹, Ile³⁰⁹, Ile³¹⁰, Arg³¹¹, Pro³¹², Asp³¹³, Ser³¹⁴, Gly³¹⁵, Pro³¹⁷, Ile³⁵¹, Gln³⁵², Gly³⁵³, Asp³⁵⁴, Gly³⁵⁵, Val³⁵⁶, Asp³⁵⁷, Thr³⁶⁰, Phe³⁸⁰, Gly³⁸¹, Ser³⁸², Gly³⁸³, Gly³⁸⁴, Gly³⁸⁵, Leu³⁸⁶, Leu³⁸⁷, Gln³⁸⁸ and Lys³⁸⁹, and in a second monomer of the dimer, Thr¹⁵, Asp¹⁶, Ser¹⁷, Tyr¹⁸, Lys¹⁹, Val²⁰, Thr²¹, His²², Gln²⁵, Arg⁴⁰, His⁹⁰, Phe⁹¹, Glu¹⁴⁹, Thr¹⁵⁰, Val¹⁵³, Trp¹⁵⁶, Le³⁹⁰, Thr³⁹¹, Arg³⁹², Asp³⁹³, Leu³⁹⁴, Asn³⁹⁶, Cys³⁹⁷, Ser³⁹⁸, Phe³⁹⁹, Lys⁴⁰⁰, Lys⁴¹⁵, Pro⁴¹⁷ and Lys⁴²³.

49. A method in accordance with claim 41, wherein the candidate compound predicted to form at least one bond with an NMPRTase is a candidate compound predicted to form at least one bond with at least one amino acid defining a inhibitor-binding pocket, wherein the at least one amino acid is selected from the group consisting of, in a first monomer of a human NMPRTase dimer, Thr¹⁵, Asp¹⁶, Ser¹⁷, Tyr¹⁸, Lys¹⁹, Val²⁰, Thr²¹, His²², Gln²⁵, His⁹⁰, Phe⁹¹, Asn¹⁴⁶, Glu¹⁴⁹, Thr¹⁵⁰, Val¹⁵³, Arg³⁹², Phe³⁹⁹ and Lys⁴¹⁵, and in the second monomer of the dimer, Leu¹⁷², Leu¹⁷⁶, Leu¹⁸³, Asp¹⁸⁴, Gly¹⁸⁵, Leu¹⁸⁶, Glu¹⁸⁷, Tyr¹⁸⁸, Lys¹⁸⁹, Leu¹⁹⁰, His¹⁹¹, Asp¹⁹², Phe¹⁹³, Gly¹⁹⁴, Tyr¹⁹⁵, Arg^{196,} Gly¹⁹⁷, Phe²¹⁵, Lys²¹⁶, Gly²¹⁷, Thr²¹⁸, Asp²¹⁹, Thr²²⁰, Val²²¹, Gly²³⁹, Tyr²⁴⁰, Ser²⁴¹, Val²⁴², Pro²⁴³, Ala²⁴⁴, Ala²⁴⁵, Glu²⁴⁶, His²⁴⁷, Val²⁷², Pro²⁷³, Val²⁷⁴, Ser²⁷⁵, Val²⁷⁶, Val²⁷⁷, Ser²⁷⁸, Arg³⁰², Ser³⁰³, Thr³⁰⁴, Gln³⁰⁵, Ala³⁰⁶, Pro³⁰⁷, Leu³⁰⁸, Ile³⁰⁹, Ile³¹⁰, Arg³¹¹, Pro³¹², Asp³¹³, Leu³²⁵, Leu³⁴³, Leu³⁴⁴, Pro³⁴⁵, Pro³⁴⁶, Tyr³⁴⁷, Leu³⁴⁸, Arg³⁴⁹, Val³⁵⁰, Ile³⁵¹, Gln^{352,} Gly³⁵³, Asp³⁵⁴, Met³⁶⁸, Ser³⁷⁴, Ile³⁷⁵, Glu³⁷⁶, Asn³⁷⁷, Ile³⁷⁸, Ala³⁷⁹, Phe³⁸⁰, Gly³⁸¹, Ser³⁸², Gly³⁸³ and Gly³⁸⁴.

50. A method in accordance with claim 41, wherein the candidate compound predicted to form at least one bond with an NMPRTase is a candidate compound predicted to form at least one bond with at least one amino acid defining a inhibitor-binding domain, wherein the at least one amino acid is selected from the group consisting of, in a first monomer of a human NMPRTase dimer, Asp¹⁶ and Tyr¹⁸, and in a second monomer of the dimer, Tyr¹⁸⁸, Lys¹⁸⁹, His¹⁹¹, Phe¹⁹³, Arg¹⁹⁶, Asp²¹⁹, Val²⁴², Pro²⁴³, Ala²⁴⁴, Pro²⁷³, Ser²⁷⁵, Pro³⁰⁷, Ile³⁰⁹, Arg³¹¹, Arg³⁴⁹, Val³⁵⁰, Ile³⁵¹, Glu³⁷⁶, Asn³⁷⁷, Ile³⁷⁸ and Ala³⁷⁹.

51. A method in accordance with claim 41, wherein the candidate compound predicted to form at least one bond with an NMPRTase is selected from the group consisting of an antibody, a peptide, an aptamer, an avimer, and an organic molecule having a molecular weight of at least about 80 daltons up to about 2000 daltons.

52. A computer-readable medium encoded with one or more sets of three dimensional coordinates of one or more NMPRTases as represented in at least one table selected from the group consisting of Table 1, Table 2 and Table 3, wherein, using a graphical display software program, the three dimensional coordinates create an electronic file that can be visualized on a computer capable of representing the electronic file as a three dimensional image.

53. A computer-readable medium encoded with one or more sets of three dimensional coordinates of one or more three dimensional structures wherein each structure substantially conforms to the three dimensional coordinates represented in a table selected from the group consisting of Table 1, Table 2, Table 3 and a combination thereof, wherein, using a graphical display software program, the set of three dimensional coordinates create an electronic file that can be visualized on a computer capable of representing said electronic file as one or more three dimensional images.

54. A method for designing a drug which interferes with the activity of a nicotinamide phosphoribosyltransferase (NMPRTase), the method comprising:
(a) providing on a digital computer a three-dimensional structure of a NMPRTase;
(b) using software comprised by the digital computer to design a chemical compound which is predicted to bind to the NMPRTase;
(c) obtaining the chemical compound; and
(d) evaluating the chemical compound for an ability to interfere with an activity of the NMPRTase.

55. A method according to claim 54, wherein the chemical compound is designed by computational interaction with reference to a site of a three-dimensional structure of an NMPRTase or an NMPRTase-ligand complex, wherein three-dimensional structure comprises atomic coordinates that substantially conform to atomic coordinates set forth in a table selected from the group consisting of Table 1, Table 2, Table 3 and a combination thereof.

56. A method in accordance with claim 54, wherein obtaining the chemical compound comprises synthesizing the candidate compound.

57. A method for designing a drug which enhances activity of a nicotinamide phosphoribosyltransferase (NMPRTase), the method comprising:
(a) providing on a digital computer a three-dimensional structure of an NMPRTase complexed with at least one NMPRTase ligand;
(b) using software comprised by the digital computer to design a chemical compound which is predicted to bind to the NMPRTase;
(c) obtaining the chemical compound; and
(d) evaluating the chemical compound for an ability to enhance activity of the NMPRTase.

58. A method according to claim 57, wherein the chemical compound is designed by computational interaction with reference to a site of a three-dimensional structure of an NMPRTase or an NMPRTase-ligand complex, wherein three-dimensional structure comprises atomic coordinates that substantially conform to atomic coordinates set forth in a table selected from the group consisting of Table 1, Table 2, Table 3 and a combination thereof.

59. A method in accordance with claim 58, wherein obtaining the chemical compound comprises synthesizing the candidate compound.

60. A method for generating a model of a three dimensional structure of NMPRTase, the method comprising:
(a) providing an amino acid sequence of a known NMPRTase and an amino acid sequence of a target NMPRTase;
(b) identifying structurally conserved regions shared between the known NMPRTase and the target NMPRTase; and
(c) assigning atomic coordinates from the conserved regions to the target NMPRTase.

61. A method in accordance with claim 60, wherein the known NMPRTase has a three dimensional structure described by atomic coordinates that substantially conform to atomic coordinates set forth in a table selected from the group consisting of Table 1, Table 2, Table 3 and a combination thereof.

62. A method for determining a three dimensional structure of a target NMPRTase, the method comprising:
(a) providing an amino acid sequence of a target NMPRTase, wherein the three dimensional structure of the target NMPRTase is not known;
(b) predicting the pattern of folding of the amino acid sequence in a three dimensional conformation using a fold recognition algorithm; and
(c) comparing the pattern of folding of the target structure amino acid sequence with the three dimensional structure of a known NMPRTase.

63. A method in accordance with claim 62, wherein the known NMPRTase has a sequence set forth in SEQ ID NO: 1 or SEQ ID NO: 2.

64. A method in accordance with claim 62, wherein the three dimensional structure of a known NMPRTase is described by atomic coordinates that substantially conform to atomic coordinates set forth in a table selected from the group consisting of Table 1, Table 2, Table 3 and a combination thereof.

65. A method in accordance with claim 41, wherein the candidate compound predicted to form at least one bond with an NMPRTase is a candidate compound predicted to promote pi-stacking interactions between Phe¹⁹³ and Tyr^{18′}.
